(19) United States
(12) Patent Application Publication
(10) Pub. No.: US 2024/0399024 A1
(43) Pub. Date: Dec. 5, 2024

(54) MICROPARTICLE TISSUE SCAFFOLD COMPOSITIONS, APPARATUSES, METHODS OF PREPARATION, AND USES THEREOF

(71) Applicant: BIO-CHANGE LTD., MP Carmel Beach (IL)

(72) Inventors: Ishay ATTAR, Yokneam (IL); Sinik KEREN, Yokneam (IL); Shani COHEN, Yokneam (IL)

(21) Appl. No.: 18/572,312

(22) PCT Filed: Jun. 21, 2022
(86) PCT No.: PCT/IB2022/000349

§ 371 Date: Dec. 20, 2023

Related U.S. Application Data
(60) Provisional application No. 63/212,993, filed on Jun. 21, 2021.

(51) Int. Cl. A61L 27/22; A61L 27/24; A61L 27/48; A61L 27/52; A61L 27/54; A61L 27/56; C12N 5/00

(52) U.S. Cl. CPC A61L 27/227; A61L 27/222; A61L 27/24; A61L 27/48; A61L 27/52; A61L 27/54; A61L 27/56; C12N 5/0075; A61L 2400/18; A61L 2430/34; C12N 2533/54; C12N 2537/10

(57) ABSTRACT

Embodiments of the present disclosure relate to particles. compositions. tissue scaffolds. apparatuses. and uses and methods thereof for body contouring. tissue engineering, regenerative medicine, aesthetic dermatology, and reconstructive procedures or surgery.

### CROSS-REFERENCE TO RELATED APPLICATIONS

This application claims the benefit of priority under 35 U.S.C. § 119 (e) to U.S. Provisional Application No. 63/212,993, filed Jun. 21, 2021, the contents of which are herein incorporated by reference in its entirety.

### FIELD OF INVENTION

Embodiments of the present disclosure relate to injectable microparticle scaffold compositions for reconstructive use.

### BACKGROUND OF INVENTION

A high incidence of soft tissue damage and loss occurs among patients caused by acute tissue injuries, disease, or elective procedures, such as breast lumpectomy or tumor removal. The results of these kinds of injuries and treatment procedures and surgeries are in most cases, unpleasing aesthetically, leading to scars and deformed tissue, which often require sequential subsequent surgeries for reconstruction. Additional defects can be attributed to loss of skin proteins, flexibility, and smoothness as a result of aging processes.

Current treatment options available rely on degradable fillers or fat grafting, which is a surgical procedure with a low success rate and is associated with patient morbidity. Most biodegradable dermal fillers suffer either from a temporary effect or from unnatural outcomes. Permanent fillers such as silicon or polymethyl-methacrylate microspheres (PMMA) are now rarely used & considered unsafe as they are known to lead to serious adverse clinical outcomes, including recurrent hematomas, edema, hypertrophic scarring, nodule formation, and cancer in some instances.

Accordingly, there is a need for an appropriate agent that can assist in reconstruction of lost or decreased soft tissue volume, rejuvenation, or substitution of defective or absent tissue.

Gelatin provides for an attractive implantable biomaterial for tissue engineering and regenerative medicine. In order to obtain a cross-linked structure, UV light applied to commercially available gelatin modified with pendant methacrylate groups, e.g., gelatin methacrylate (GelMA) fabricates crosslinked hydrogels using radical polymerization. In this example, crosslinking leaves behind toxic free radicals and has sub-optimal biocompatibility.

Therefore, there is a need for a safe, inexpensive, cost-efficient implantable tissue support for reconstructive uses, for example, body contouring and bio-stimulation. Moreover, the tissue support should not induce a harmful immune system response (i.e., lack of immunogenicity). It should also be degradable within a short timeframe not to risk granulomas.

Yet another need is to enhance the seeding and survival of cell therapies injected into tissues. Those cells usually do not retain very well and there is a need to assist them by providing a supportive biocompatible scaffold, to serve as an initial and intermediate bed for their attachment in the treated tissue.

### SUMMARY

One object of the present disclosure is to provide an improved microparticle porous scaffold composition, optionally crosslinked enzymatically, and optionally injectable into the body or through injectors with needle.

Another object can be directed to a plurality of microparticles, comprising: a cross-linked protein, where the cross-linked protein comprises at least one RGD (Arg-Gly-Asp) motif; where the plurality of microparticles is essentially or substantially cross-linker-free; and where the plurality of microparticles is water insoluble. One aspect of the plurality of microparticles is that the cross-linked protein can be selected from a group consisting of: gelatin, collagen, casein, elastin, tropoelastin, albumin, engineered protein thereof, and the like, or any combinations thereof; or in other aspects, the cross-linked protein is selected from: non-recombinant gelatin, recombinant gelatin, non-recombinant collagen, recombinant collagen, engineered protein or synthetic protein thereof, any engineered polymer with a RGD motif linked thereto, and the like, or combinations thereof. Some aspects provide such plurality of particles of the disclosure composed of: lyophilized foam particles; particle sizes (e.g., dry or wet particles) selected from: 0.1 μm-2000 μm (e.g., 40 μm-100 μm; 60 μm-90 μm); at least two different particle sizes selected from: 0.1 μm-2000 μm (e.g., 40 μm-100 μm; 60 μm-90 μm); a mean particle size selected from 0.1 μm-2000 μm (e.g., 30 μm-500 μm; 40 μm-100 μm; 60 μm-90 μm), or combinations thereof.

In other objects of the disclosure, a method of preparing the plurality of microparticles described here, comprise: (a) mixing a cross-linkable protein solution and a cross-linker solution, where the cross-linkable protein solution comprises dissolving a cross-linkable protein or engineered polymer comprising at least one RGD (Arginine-Glycine-Aspartate (Arg-Gly-Asp)) motif or linked thereto in a liquid; and where the cross-linker solution comprises dissolving a cross-linker in a liquid; (b) forming a cross-linked foam comprising the mixed cross-linkable protein solution and cross-linker solution of (a); (c) removing the cross-linker from the cross-linked foam of (b) to form a cross-linker-free foam; and (d) reducing in size; the formed cross-linked foam of (b), the cross-linker-free foam of (c), or combinations of the formed cross-linked foam of (b) and the cross-linker-free foam of (c), to form a plurality of microparticles comprising size-reduced cross-linked foam of (b) and/or size-reduced cross-linker-free foam of (c). Another aspect of the method provides the mixing of (a) having steps of: (a1) preparing the cross-linkable protein solution, by: adding a cross-linkable protein to a liquid (e.g., water, saline, PBS) at 50° C. while stirring or continuously stirring; and dissolving the cross-linkable protein to form the cross-linkable protein solution; (a2) preparing the cross-linker solution, by: adding a cross-linker to a liquid (e.g., water, saline, PBS) at 25° C. while stirring or continuously stirring: and dissolving the cross-linker to form the cross-linker solution. In some aspects of the method disclosed here, the cross-linked foam of (b) is enzymatically cross-linked, where the cross-linker is transglutaminase (e.g., microbial transglutaminase). Further aspects of the method of the disclosure of preparing the plurality of microparticles, where the formation of the cross-linked foam of (b) comprises: whipping or agitating to acrate or stirring with gas or air (e.g., argon, carbon dioxide, helium, hydrogen, krypton, methane, neon, nitrogen, oxygen, ozone, water vapor, xenon) or without. In some aspects, the method comprises stirring the cross-linker solution of (a) without gas or air to form a confluent crosslinked protein (b) that is not a foam, which can be further treated and reduced in size in the same manner as that performed for the foam of (c)-(g). The cross-linkable protein solution of (a) while adding the cross-linker solution of (a) at 37° C. to form the cross-linked foam of (b). Whipping allows for aeration in the cross-linked foam formation. In some embodiments, formation of the cross-linked foam of (b) can occur by stirring or mixing without gas or air. In yet another aspect of the method, the reducing of (d), comprises cutting (e.g., dicing, chopping, meshing) the formed cross-linked foam of (b) into large pieces of foam having a size of 0.2 mm-20 mm (e.g., 1 mm-19 mm; 2 mm-18 mm; 3 mm-17 mm; 4 mm-16 mm; 5 mm-15 mm; 6 mm-14 mm; 7 mm-13 mm; 8 mm-12 mm; 9 mm-11 mm); 0.5 mm or greater (e.g., 0.6 mm; 0.7 mm; 0.8 mm; 0.9 mm; 1 mm; 2 mm; 3 mm; 4 mm; 5 mm; 6 mm; 7 mm; 8 mm; 9 mm; 10 mm; 11 mm; 12 mm; 13 mm; 14 mm; 15 mm; 16 mm; 17 mm; 18 mm; 19 mm; 20 mm); 20 mm or less (e.g., 19.5 mm; 18.5 mm; 17.5 mm; 16.5 mm; 15.5 mm; 14.5 mm; 13.5 mm; 12.5 mm; 11.5 mm; 10.5 mm; 9.5 mm; 8.5 mm; 7.5 mm; 6.5 mm; 5.5 mm; 4.5 mm; 3.5 mm; 2.5 mm; 1.5 mm). Some aspects of the method further directed to removing of (c) can comprise: removing the cross-linker or crosslinking enzyme by, for example, washing the cross-linked foam of (b), where the cross-linked foam of (b) is reduced in size by cutting (e.g., diced, chopped, meshed) into pieces, where washing occurs by agitating the pieces of cross-linked foam in a liquid at 45° C.-55° C. (e.g., 50° C.) to form washed foam pieces; and sieving the washed foam pieces of (c1) on a mesh sieve (e.g., one or more mesh sieves; 35 US Mesh #-5000 US Mesh #; 2.5 mm-500 mm; 0.5 mm), thereby forming cross-linker-free foam pieces (e.g., 0.2 mm-20 mm).

Another object of the method can be directed to further comprising: (e) freezing the cross-linker-free foam of (c) or plurality of particles of (d); (f) drying (e.g., lyophilizing, freeze-drying, oven drying, room temperature drying. ambient drying) the frozen cross-linker-free foam of (c); and (g) reducing in size the lyophilized cross-linker-free foam of (f) to form a plurality of cross-linked foam particles. The plurality of cross-linked foam particles of the method comprises a particle size (e.g., dry or wet particles) of 0.1 μm-2000 μm (e.g., 5 μm-150 μm; 40 μm-100 μm; 60 μm-90 μm). In one aspect of the method, the cross-linkable protein can be selected from: gelatin, collagen, casein, elastin, tropoelastin, albumin, engineered protein thereof, and the like, or any combinations thereof, where the cross-linkable protein can further be selected from the group consisting of: non-recombinant gelatin, recombinant gelatin, non-recombinant collagen, recombinant collagen, and the like, or engineered polymer comprising at least one RGD motif or linked thereto, and the like, or any combinations thereof. Some aspects can be directed to the cross-linker, where the cross-linker is an enzyme, such as but not limited to, transglutaminase or oxidative enzyme. Non-limiting examples of such cross-linkers; natural transglutaminase, modified transglutaminase, recombinant transglutaminase, microbial transglutaminase (mTG), tissue transglutaminase (tTG), keratinocyte transglutaminase, epidermal transglutaminase, prostate transglutaminase, neuronal transglutaminase, human transglutaminase, Factor XIII, natural oxidative enzyme, modified oxidative enzyme, lysyl oxidase, tyrosinase, laccase, peroxidase, and the like, or any combinations thereof. Moreover, in another aspect of the methods of the disclosure, the freezing of (c) can occur at −18° C.-25° C. for a minimum of 2 hours (e.g., 3 hours, 4 hours, 5-25 hours); the lyophilizing of (f) can occur at −50° C.∓10° C., 0.01 mbar-0.1 mbar (e.g., 0.04 mbar-0.05 mbar), and 24 hours-96 hours (e.g., 48 hours).

Yet another embodiment is to stir the protein solutions of (a) without gas or air to form a confluent crosslinked protein (b) that is not a foam, which can be further treated and reduced in size in the same manner as that performed for the foam of: (c) removing the cross-linker from the cross-linked foam or block of (b) to form a cross-linker-free foam or block; and reducing in size, the formed cross-linked foam or block of (b); (e) freezing the cross-linker-free foam or block of (c) or plurality of particles of (d); (f) lyophilizing the frozen cross-linker-free foam or block of (e); (g) reducing in size the lyophilized cross-linker-free foam of (f) to form a plurality of cross-linked foam particles.

A further aspect of the methods of the disclosure are directed to the reducing in size of (g) that comprises: pulverizing the lyophilized cross-linker-free foam of (e) to form the plurality of cross-linker-free foam particles; separating by size, the plurality of cross-linker-free foam particles. The reducing in size can result in a plurality of cross-linker-free foam particles having a particle size of 0.1 μm-2000 μm (dry or wet particle size). Another aspect of the methods of the disclosure provides for the separating by size of the pulverized lyophilized cross-linker-free foam of (e), by sieving the plurality of cross-linker-free foam particles to generate the plurality of cross-linked foam particles having one or more, or at least two different particle size ranges.

A further object of the disclosure can be directed to a composition, comprising: (a) the plurality of microparticles of the disclosure; and (b) a carrier, where the cross-linked protein is selected from: gelatin, collagen, casein, elastin, tropoelastin, albumin, engineered protein thereof, and the like, or any combinations thereof: or the cross-linked protein is selected from: non-recombinant gelatin, recombinant gelatin, non-recombinant collagen, recombinant collagen, and the like, or engineered polymer comprising at least one RGD motifs or any combinations thereof. Further aspects can provide for a carrier of the composition that is a hydrogel, where the carrier can be selected from: gelatin; collagen; alginate; hyaluronic acid; carboxymethyl cellulose; poly(ethylene oxide) (PEO); poly(vinyl alcohol) (PVA); poly (propylene fumarate) (PPF); polyethylene glycol (PEG); and the like, or any combinations thereof; or the carrier is selected from: gelatin (e.g., non-crosslinked, crosslinked, in situ crosslinking); collagen (e.g., non-crosslinked, crosslinked); alginate (e.g., non-crosslinked, crosslinked); hyaluronic acid (e.g., non-crosslinked, crosslinked); PEG; carboxymethyl cellulose; and the like, or combinations thereof. Aspects of these compositions also provide a concentration of the plurality of microparticles in the carrier of: 1 mg/ml or greater (e.g., 10 mg/ml; 20 mg/ml; 30 mg/ml; 40 mg/ml; 50 mg/ml; 60 mg/ml; 70 mg/ml; 80 mg/ml; 90 mg/ml; 100 mg/ml; 110 mg/ml; 120 mg/ml; 130 mg/ml; 140 mg/ml; 150 mg/ml; 200 mg/ml; 300 mg/ml); 300 mg/ml or less (e.g., 290 mg/ml; 280 mg/ml; 270 mg/ml; 260 mg/ml; 250 mg/ml; 240 mg/ml; 230 mg/ml; 220 mg/ml; 210 mg/ml; 200 mg/ml; 190 mg/ml; 180 mg/ml; 170 mg/ml; 160 mg/ml; 155 mg/ml; 145 mg/ml; 135 mg/ml; 125 mg/ml; 115 mg/ml; 105 mg/ml; 95 mg/ml; 85 mg/ml; 75 mg/ml; 65 mg/ml; 55 mg/ml; 45 mg/ml; 35 mg/ml; 25 mg/ml; 15 mg/ml; 5 mg/ml); or 1 mg/ml-300 mg/ml (e.g., 2 mg/ml-295 mg/ml; 4 mg/ml-285 mg/ml; 6 mg/ml-275 mg/ml; 8 mg/ml-265 mg/ml; 12 mg/ml-255 mg/ml; 14 mg/ml-245 mg/ml; 16 mg/ml-235 mg/ml; 18 mg/ml-225 mg/ml; 22 mg/ml-215 mg/ml; 24 mg/ml-205 mg/ml; 26 mg/ml-195 mg/ml; 28 mg/ml-185 mg/ml; 32 mg/ml-175 mg/ml; 34 mg/ml-165 mg/ml; 36 mg/ml-153 mg/ml; 38 mg/ml-143 mg/ml; 42 mg/ml-133 mg/ml; 52 mg/ml-123 mg/ml; 62 mg/ml-113 mg/ml; 72 mg/ml-103 mg/ml; 82 mg/ml-93 mg/ml).

Another object of the disclosure provide a tissue scaffold, comprising: the plurality of microparticles of the disclosure, and in some aspects, further comprises a hydrogel carrier, where the hydrogel carrier is selected from: gelatin, collagen, alginate, hyaluronic acid, carboxymethyl cellulose, and the like, or any combinations thereof. In some other aspects, the tissue scaffold is configured as a foam, and the cross-linked protein microparticles comprise at least one different particle sizes, where the particle size can be 0.1 μm-2000 μm.

Yet a further object of the disclosure can be directed to an apparatus, comprising the composition of the disclosure comprising a plurality of microparticles and a carrier, where the apparatus is, in some aspects, a syringe, a cartridge, or a vial. Another aspect provides a syringe comprising a needle or a cannula selected from 14 gauge to 39 gauge (e.g., 25 gauge-30 gauge, 27 gauge-30 gauge). In one aspect of the apparatus, the apparatus is sterilizable or configured for sterilization.

In another object of the disclosure, use of the composition comprising a plurality of microparticles and a carrier, and/or the plurality of microparticles of the disclosure, can be for body contouring in a subject (either human or animal). An aspect of the use provides for body contouring selected from: soft tissue reconstruction, volume restoration, breast augmentation, biostimulation (of cells, e.g., of skin), and the like, or combinations thereof. In some aspects, biostimulation can be selected from: fibroblast stimulation, collagen production stimulation, neo-collagenesis, tissue regrowth, wound closure and the like, or combinations thereof. Another aspect of the use is directed to the composition of the disclosure and/or the plurality of the microparticles of the disclosure, where the composition and/or the plurality of microparticles are configured in the apparatus described here, which can be, for example, a syringe, a cartridge, or a vial.

One object of the disclosure also provides for a method of treating a subject in need of body contouring, comprising administering the composition of the disclosure at a site of the subject in need of body contouring, where administration in one aspect is by injection. Another aspect provides for administration of the composition of the disclosure which results in: (a) stimulating fibroblasts; (b) stimulating collagen production; (c) inducing neo-collagenesis; (d) inducing tissue regrowth; (e) providing a tissue scaffold; or the like, or (f) any combinations thereof.

### BRIEF DESCRIPTION OF FIGURES

FIG. 1 shows a graphical representation of the average activity of crosslinker, mTG, in various microparticle batches, where the amount of mTG used for gelatin crosslinking was 3 g-10 g. P1: assay positive control; mTG (1:100); positive control; Y-axis; mTG average activity; X-axis; microparticle batches and positive controls.

FIGS. 2A-2D show a representative histopathological evaluation of a subcutaneous area implanted with the composition of the disclosure, 30 days following injection, using Masson's Trichrome (MT) staining. The scales for each are as follows: 1000 μm (FIG. 2A); 200μ (FIG. 2B); and 50μ (FIGS. 2C-2D). Black arrows indicate the implanted composition of the disclosure. Gray arrows indicate neo-collagenesis. The white arrow shows the interaction between infiltrating fibroblasts and the scaffold.

FIG. 3 demonstrates a linear correlation between particles size to injection force, using a 1 ml syringe with 27 gauge (G) needle.

FIG. 4 shows an exemplary Scanning Electron Microscopy (SEM) image of particles greater than 0.1 μm (i.e., 100 nm) (e.g., 104 nm; 105 nm; 112 nm; 145 nm; 150 nm; 275 nm).

FIG. 5 shows an exemplary SEM image of microparticles having a particle size range of 60 μm-100 μm (e.g., 75.69 μm; 88.38 μm; 91.56 μm; 99.68 μm).

FIG. 6A shows an exemplary light microscopy image of particles up to 2000 μm. The particles were hydrated before imaging (scale 500 μm). FIG. 6B shows dry particle sizes of: 558 μm, 862 μm, and 986 μm (scale 200 μm). FIG. 6C shows a wet particle having a particle size of 1808 μm (scale 500 μm).

FIG. 7A shows dry gelatin microparticles (scale 100 μm). FIG. 7B shows hydrated or wet gelatin microparticles (scale 100 μm).

FIG. 8 shows a graphical representation of size distribution of dry and hydrated microparticles. Dry microparticles (Left Column): Particle sizes: 70 μm-170 μm. Hydrated microparticles (Right Column): 90 μm-310 μm.

FIG. 9 shows a representative frequency sweep graph, displaying on the Y-axis the storage modulus G′ (Pa)Δ, Loss modulus G″ (Pa)□, and the complex viscosity η*(Pa·s) O, of 0.75% gelatin carrier at 6° C. to the frequency f(Hz) on the X-axis.

FIG. 10 shows a representative size distribution of exemplary foam microparticles of the sample (8 gr mTG), where the 96% ethanol sample (circle) peaked at 14 volume % at a size of 80 μm, the DDW instant sample (diamond) peaked at 10 volume % at a size of 120 μm; and the DDW at 24 hours sample (square) peaked at 11 volume % at a size of 140 μm. See, TABLE 6.

FIG. 11 shows the injectability (N) of an exemplary formulation of crosslinked gelatin foam microparticles with different saline volumes (1.5 ml; 2 ml; 3 ml; 4 ml).

FIG. 12 shows representative histologic photographs of implants stained with H&E (Hematoxylin and Eosin which stains cell nuclei a purplish blue, and the extracellular matrix and cytoplasm pink) and MT, Mason Trichrome (produce red keratin, muscle fibers and implant, blue collagen and bone, light red or pink cytoplasm, and dark brown to black cell nuclei) in pig and rat skin at 7-, 30-, and 180-days post-implantation (H&E-pig Day 7 and Day 30, Rat Day 7 and Day 30, and MT-pig Day 180), the arrows showing sites of the implanted composition of the disclosure.

FIG. 13 shows representative histologic photographs of implants stained with H&E (Hematoxylin and Eosin which stains cell nuclei a purplish blue, and the extracellular matrix and cytoplasm pink) and Mason Trichrome (produce red keratin, muscle fibers and implant, blue collagen and bone, light red or pink cytoplasm, and dark brown to black cell nuclei) at 7-, 30-, and 180-days post-implantation (H&E-pig Day 7 and Mason Trichrome pig Day 30 and Day 180, Rat Day 7 and Day 30) showing the implanted formulation (black arrows) of new collagen fibers stained in blue (white arrows).

FIG. 14 shows an SDS-PAGE of 1 mg/ml FPs prepared in water. Collagenase was added to the suspension to degrade the FPs. Molecular weight marker (M); microbial transglutaminase (7 μg protein in 20 μl) (1); gelatin (10 μg protein in 20 μl) (2); collagenase (1.7 U in 20 μl) (3); foam particles (FPs) (degraded with collagenase, 10 μg protein in 20 μl) (4).

FIG. 15 shows a calibration curve of arginine. R² value of 0.999 indicates high linearity. Arginine concentration (μg/ml) (X-axis) to emission intensity (Y-axis).

FIG. 16 shows fluorescence emissions spectra of free arginine, raw materials, and crosslinked gelatin particles.

FIG. 17 shows RGD quantification in raw materials non-crosslinked gelatin and crosslinked gelatin particles (i.e., FPs and confluent particles).

FIG. 18 shows the amount of RGD sequences or motifs (μg/mg) (Y-axis) on FPs with different crosslinked gelatin particle size ranges (X-axis) of less than 63 μm; 63 μm-99 μm; and more than 99 μm.

FIG. 19 shows the amount of RGD (ug/mg) (Y-axis) in relation to various weight ratios of gelatin; mTG, gelatin, and microbial transglutaminase (mTG).

FIG. 20A and FIG. 20B show light microscopy images of human induced pluripotent stem (iPS) cells grown on foam particle microcarriers of the disclosure differentiated into cardiomyocytes. FIG. 20A has a scale of 50 μm and FIG. 20B has a scale 200 μm.

FIG. 21A and FIG. 21B show light microscopy images of foam particles (FPs) produced from foam crosslinked gelatin fibers. Scale 100 μm.

### DETAILED DESCRIPTION

Detailed embodiments of the present disclosure are described here; however, it is to be understood that the disclosed embodiments are merely illustrative of the invention that can be embodied in various forms. In addition, each of the examples given in connection with the various embodiments of the invention is intended to be illustrative, and not restrictive.

Porous and biodegradable polymer scaffolds can be utilized as a structural supporting matrix or as cell adhesive substrates. It is an object of the disclosure to provide a safe, non-toxic, inexpensive or low cost, implantable tissue support that does not induce an immune response or lacks immunogenicity. In one instance, the implantable tissue support of the disclosure is synthetic and/or lacks or is essentially free of non-human components. Using a material with inherent cell-binding elements can improve the performance of implants by allowing direct cell attachment and local remodeling. A tripeptide motif (e.g., RGD (Arginine (Arg)-Glycine (Gly)-Aspartate (Asp))) is found within extracellular matrix proteins, such as but not limited to, bone sialoprotein, collagen, fibrinogen, fibronectin, gelatin, laminin, osteopontin, and vitronectin, facilitates cell adhesion, cell membrane binding, and cell attachment. The RGD motif is an integrin-binding domain within ECM proteins. For example, gelatin, derived from collagen, contains the RGD motif that is useful for cell adhesion.

### Particles

In various embodiments of the disclosure, microparticles or a plurality of microparticles; their methods of preparation; compositions comprising the plurality of microparticles; apparatuses, such as syringes or vials, comprising the compositions of the disclosure; scaffolds or tissue scaffolds comprising the plurality of microparticles or compositions of the disclosure; uses of the disclosed plurality of microparticles or compositions of the disclosure; and methods of treating a subject by administering the plurality of microparticles or compositions of the disclosure are provided here.

As used herein, the term “subject” refers to any organism to which a composition in accordance with the disclosure can be administered, e.g., for experimental, diagnostic, prophylactic, and/or therapeutic purposes. Typical subjects include any animal (e.g., mammals such as mice, rats, rabbits, dogs, cats, non-human primates, and humans, etc.). A subject in need thereof is typically a subject for whom it is desirable to treat a disease, disorder, or condition as described herein. For example, a subject in need thereof can seek or be in need of treatment, require treatment, be receiving treatment, can be receiving treatment in the future, or a human or animal that is under care by a trained professional for a particular disease, disorder, or condition. In some embodiments, the subject is in need of body contouring, including but not limited to: soft tissue reconstruction, volume restoration, breast augmentation, biostimulation (of cells, e.g., of skin), and the like, or combinations thereof. In some embodiments, biostimulation can be selected from: fibroblast stimulation, collagen production stimulation, neo-collagenesis, tissue regrowth, wound closure and the like, or combinations thereof.

One embodiment of the disclosure is directed to a microparticle or a plurality of microparticles having: a cross-linked protein, where the protein of the cross-linked protein comprises at least one RGD (Arg-Gly-Asp) motif, which conveys, inter alia, cell adhesion properties. In some embodiments, the cross-linked protein having several RGD motifs, but at least one RGD motif, can be sufficiently and/or more advantageously exposed by reducing in particle size and increasing the surface area of the microparticles. The microparticle or the plurality of microparticles described here comprises a cross-linked protein that has an RGD motif in an amount of 0.1 μg/mg-50 μg/mg (e.g., 0.2 μg/mg-45 μg/mg; 0.3 μg/mg-40 μg/mg; 0.4 μg/mg-35 μg/mg; 0.5 μg/mg-30 μg/mg; 0.6 μg/mg-25 μg/mg; 0.7 μg/mg-20 μg/mg; 0.8 μg/mg-15 μg/mg; 0.9 μg/mg-10 μg/mg; 1 μg/mg-5 μg/mg); of 0.1 μg/mg or greater (e.g., 2 μg; 4 μg; 6 μg; 8 μg; 10 μg; 12 μg; 14 μg; 16 μg; 18 μg; 20 μg; 22 μg; 24 μg; 26 μg; 28 μg; 30 μg; 32 μg; 34 μg; 36 μg; 38 μg; 40 μg; 42 μg; 44 μg; 46 μg; 48 μg; 50 μg); or of 50 μg/mg or less (e.g., 49 μg; 47 μg; 45 μg; 43 μg; 41 μg; 39 μg; 37 μg; 35 μg; 33 μg; 31 μg; 29 μg; 27 μg; 25 μg; 23 μg; 21 μg; 19 μg; 17 μg; 15 μg; 13 μg; 11 μg; 9 μg; 7 μg; 5 μg; 3 μg; 1 μg; 0.9 μg; 0.7 μg; 0.5 μg; 0.3 μg; 0.1 μg).

The microparticle or plurality of microparticles, including the cross-linked protein, is cross-linker-free or essentially cross-linker-free, where “cross-linker-free” as used here means absent cross-linkers or containing nominal amounts of cross-linkers, which can be present, but have no effect on the function or use of the plurality of microparticles or cross-linked protein. The cross-linked protein can be stabilized into, for example, a foam, into a confluent hydrogel, or into fibers, where the cross-linking occurred by enzymatic crosslinking. In some embodiments, the enzymatic crosslinking was performed using an enzyme, but subsequently removed by, for example, washing the enzyme out of the particles or inactivating the cross-linker or crosslinking enzyme. One embodiment comprises the use of a transglutaminase enzyme for cross-linking the protein of the cross-linked protein, one upon completion of cross-linking, the enzyme is washed out of the cross-linked protein(s). In a further embodiment, the transglutaminase enzyme is or comprises a microbial transglutaminase enzyme.

Accordingly, the final microparticle or plurality of microparticles comprises cross-linked proteins that are cross-linker-free. In some aspects, the protein of the cross-linked protein of the final microparticle or plurality of microparticles comprises proteins previously cross-linked or pre-cross-linked proteins, where the cross-linked proteins have been washed to remove any cross-linkers, such that the final microparticle or plurality of microparticles comprising cross-linked proteins is cross-linker-free or essentially or substantially cross-linker-free. The protein of the cross-linked protein can be selected from, but not limited to, gelatin, collagen, casein, elastin, tropoelastin, albumin, engineered protein thereof, and the like, or any combinations thereof. Other aspects of the embodiment can be directed to proteins of the cross-linked proteins comprising: non-recombinant gelatin, recombinant gelatin, non-recombinant collagen, recombinant collagen, engineered protein thereof, engineered polymer comprising at least one RGD motif or linked thereto, and the like, or any combinations thereof. Furthermore, the microparticles or plurality of microparticles of the disclosure comprise at least one or more cross-linked proteins, where the at least one or more cross-linked proteins comprise at least one RGD (Arg-Gly-Asp) motif; where the microparticle or plurality of microparticles is cross-linker-free (i.e., absent or essentially absent of cross-linker(s)); and the microparticle or plurality of microparticles is water-insoluble or essentially water-insoluble. Some embodiments of the disclosure are directed to a plurality of microparticles that are pre-crosslinked, water insoluble, and cross-linker-free, and are not water soluble.

Another embodiment is directed to a plurality of microparticles of the disclosure, where the microparticles comprise particles of foam or particles having a foam-like property, where the plurality of microparticles or foam particles comprise cross-linked proteins that are cross-linker-free. In some aspects of the embodiments of the disclosure, the cross-linked proteins are stabilized into a foam, into a confluent hydrogel, or into fibers (such as in electro-spinning), where the cross-linking occurred by enzymatic crosslinking. As used here, “foam” means a dispersion of gas bubbles in a liquid, solid, or semi-solid (e.g., gel). In other instances, disclosed here, a foam can comprise or be configured as particles. These foam particles either retain properties of a foam or are derived from foam thereby having “foam-like” properties. Moreover, the plurality of microparticles or foam particles can be composed of lyophilized particles, including lyophilized foam particles comprising cross-linked proteins that are cross-linker free. “Foam particles” (FPs) as used herein means that they originate from a stable protein foam, and are not necessarily foam in their own structure, after the pulverization. This can depend on the size of the gas bubbles in the initial cross-linker-free foam of (c) and the size of the resulting lyophilized and size-reduced particles of (g). If the gas bubbles are smaller than the particle size, they can contain closed cells of the foam; however, if the particles are smaller than the gas bubbles, then bubbles or full bubbles cannot remain enclosed in the particles. In either event, the performance and the intention of the embodiments described here is not impeded and is not to be limited to a foam structure.

One embodiment is directed to foam or foam particles that is reduced in size and comprises or is configured as particles, including microparticles, by cutting (e.g., chopping, dicing); using compression, lump breakers, pulverizers, mills (e.g., impact mills, flour mills, full-screen hammer mills, mega hammer mills, air classifying mills, jet mills, ball mills, pebble mills, rod mills); grinders (fine grinders, blade grinders), and the like, or combinations thereof. By reducing in size, the cross-linked foam to form particles, such as microparticles, allows for exposure of several RGD motifs to a large surface area, which facilitates cell adhesion and bio-stimulation. Those embodiments using at least two different particle sizes also benefit from enhanced surface area. Particle sizes can be analyzed or measured by any technique commonly known and/or used by persons of ordinary skill in the art. Non-limiting examples of such methods, techniques, or tools for measuring particle size include: Particle Size Analyzers (PSA); high definition image processing; image particle analysis (IPA) (e.g., optical microscopes, scanning electron microscopes (SEMs), transmission electron microscopes (TEMs)); dynamic image analysis (DIA); static laser light scattering (SLS, also known as laser diffraction); dynamic light scattering (DLS); acoustic spectroscopy; sieve analysis (e.g., dry sieving, wet sieving); and the like, or any combinations thereof.

In some embodiments, the plurality of microparticles, including but not limited to those derived from foam cross-linked proteins, comprises a particle size of 0.1 μm-2000 μm (e.g., 0.2 μm-1499 μm; 0.4 μm-1450 μm; 0.5 μm-1425 μm; 0.6 μm-1400 μm; 0.7 μm-1350 μm; 0.8 μm-1300 μm; 0.9 μm-1250 μm; 1 μm-1200 μm; 2 μm-1150 μm; 3 μm-1100 μm; 4 μm-1050 μm; 5 μm-1000 μm; 6 μm-950 μm; 7 μm-900 μm; 8 μm-850 μm; 9 μm-800 μm; 10 μm-750 μm; 11 μm-700 μm; 12 μm-650 μm; 13 μm-600 μm; 14 μm-550 μm; 15 μm-500 μm; 16 μm-450 μm; 17 μm-400 μm; 18 μm-350 μm; 19 μm-300 μm; 20 μm-250 μm; 25 μm-200 μm; 30 μm-150 μm; 40 μm-100 μm; 60 μm-90 μm); 0.1 μm or greater (e.g., 0.5 μm; 1 μm; 5 μm; 15 μm; 25 μm; 35 μm; 45 μm; 55 μm; 65 μm; 75 μm; 85 μm; 95 μm; 100 μm; 105 μm; 115 μm; 125 μm; 135 μm; 145 μm; 150 μm; 200 μm; 250 μm; 500 μm; 1000 μm; 2000 μm); or 2000 μm or less (e.g., 1250 μm; 1000 μm; 750 μm; 500 μm; 250 μm; 200 μm; 150 μm; 140 μm; 130 μm; 120 μm; 110 μm; 90 μm; 80 μm; 70 μm; 60 μm; 50 μm; 40 μm; 30 μm; 20 μm; 10 μm; 5 μm; 4 μm; 3 μm; 2 μm). Other embodiments directed to such plurality of microparticles comprises a mean particle size of: 0.1 μm-2000 μm (e.g., 0.2 μm-1499 μm; 0.4 μm-1450 μm; 0.5 μm-1425 μm; 0.6 μm-1400 μm; 0.7 μm-1350 μm; 0.8 μm-1300 μm; 0.9 μm-1250 μm; 1 μm-1200 μm; 2 μm-1150 μm; 3 μm-1100 μm; 4 μm-1050 μm; 5 μm-1000 μm; 6 μm-950 μm; 7 μm-900 μm; 8 μm-850 μm; 9 μm-800 μm; 10 μm-750 μm; 11 μm-700 μm; 12 μm-650 μm; 13 μm-600 μm; 14 μm-550 μm; 15 μm-500 μm; 16 μm-450 μm; 17 μm-400 μm; 18 μm-350 μm; 19 μm-300 μm; 20 μm-250 μm; 25 μm-200 μm; 30 μm-150 μm; 40 μm-100 μm; 60 μm-90 μm); 0.1 μm or greater (e.g., 5 μm; 15 μm; 25 μm; 35 μm; 45 μm; 55 μm; 65 μm; 75 μm; 85 μm; 95 μm; 100 μm; 105 μm; 115 μm; 125 μm; 135 μm; 145 μm; 150 μm; 200 μm; 250 μm; 500 μm; 1000 μm; 2000 μm); or 2000 μm or less (e.g., 1250 μm; 1000 μm; 750 μm; 500 μm; 250 μm; 200 μm; 150 μm; 140 μm; 130 μm; 120 μm; 110 μm; 90 μm; 80 μm; 70 μm; 60 μm; 50 μm; 40 μm; 30 μm; 20 μm; 10 μm; 5 μm; 4 μm; 3 μm; 2 μm). In embodiments of the disclosure, a “mean particle size” as used here means the average particle size of the plurality of microparticles. In some embodiments, a “particle size” refers to a dry particle size. Some embodiments, a “particle size” refers to a wet particle size. In some embodiments, a wet or hydrated particle has a greater particle size than a dry particle of the same dry size, by a factor of, for example, 1.4 to 2.8 with an average factor of 1.67 (1.65-1.67). See, e.g., TABLE 4.

Further embodiments of the disclosure are directed to the plurality of microparticles described here where the plurality of microparticles can comprise at least two different particle sizes. The particle sizes can be selected from any of the particle sizes disclosed here, including but not limited to: 0.1 μm-2000 μm (e.g., 0.2 μm-1900 μm; 0.3 μm-1800 μm; 0.4 μm-1700 μm; 0.5 μm-1600 μm; 0.6 μm-1500 μm; 0.7 μm-1400 μm; 0.8 μm-1300 μm; 0.9 μm-1250 μm; 1 μm-1200 μm; 2 μm-1150 μm; 3 μm-1100 μm; 4 μm-1050 μm; 5 μm-1000 μm; 6 μm-950 μm; 7 μm-900 μm; 8 μm-850 μm; 9 μm-800 μm; 10 μm-750 μm; 11 μm-700 μm; 12 μm-650 μm; 13 μm-600 μm; 14 μm-550 μm; 15 μm-500 μm; 16 μm-450 μm; 17 μm-400 μm; 18 μm-350 μm; 19 μm-300 μm; 20 μm-250 μm; 25 μm-200 μm; 30 μm-150 μm; 40 μm-100 μm; 60 μm-90 μm); 0.1 μm or greater (e.g., 0.5 μm; 1 μm; 5 μm; 15 μm; 25 μm; 35 μm; 45 μm; 55 μm; 65 μm; 75 μm; 85 μm; 95 μm; 100 μm; 105 μm; 115 μm; 125 μm; 135 μm; 145 μm; 150 μm; 200 μm; 250 μm; 500 μm; 1000 μm; 2000 μm); or 2000 μm or less (e.g., 1250 μm; 1000 μm; 750 μm; 500 μm; 250 μm; 200 μm; 150 μm; 140 μm; 130 μm; 120 μm; 110 μm; 90 μm; 80 μm; 70 μm; 60 μm; 50 μm; 40 μm; 30 μm; 20 μm; 10 μm; 5 μm; 4 μm; 3 μm; 2 μm). Other embodiments directed to the plurality of microparticles comprising at least two different particle sizes comprise a mean particle size selected from, but not limited to: 0.1 μm-2000 μm (e.g., 0.2 μm-1499 μm; 0.4 μm-1450 μm; 0.5 μm-1425 μm; 0.6 μm-1400 μm; 0.7 μm-1350 μm; 0.8 μm-1300 μm; 0.9 μm-1250 μm; 1 μm-1200 μm; 2 μm-1150 μm; 3 μm-1100 μm; 4 μm-1050 μm; 5 μm-1000 μm; 6 μm-950 μm; 7 μm-900 μm; 8 μm-850 μm; 9 μm-800 μm; 10 μm-750 μm; 11 μm-700 μm; 12 μm-650 μm; 13 μm-600 μm; 14 μm-550 μm; 15 μm-500 μm; 16 μm-450 μm; 17 μm-400 μm; 18 μm-350 μm; 19 μm-300 μm; 20 μm-250 μm; 25 μm-200 μm; 30 μm-150 μm; 40 μm-100 μm; 60 μm-90 μm); 0.1 μm or greater (e.g., 0.5 μm; 1 μm; 5 μm; 15 μm; 25 μm; 35 μm; 45 μm; 55 μm; 65 μm; 75 μm; 85 μm; 95 μm; 100 μm; 105 μm; 115 μm; 125 μm; 135 μm; 145 μm; 150 μm; 200 μm; 250 μm; 500 μm; 1000 μm; 2000 μm); or 2000 μm or less (e.g., 1250 μm; 1000 μm; 750 μm; 500 μm; 250 μm; 200 μm; 150 μm; 140 μm; 130 μm; 120 μm;

110 μm; 90 μm; 80 μm; 70 μm; 60 μm; 50 μm; 40 μm; 30 μm; 20 μm; 10 μm; 5 μm; 4 μm; 3 μm; 2 μm).

### Methods of Preparing Particles of the Disclosure

Although gelatin microspheres have been previously fabricated using various methods and techniques, including water-in-oil emulsion, electrospray, spray-drying, and microfluidic emulsification to name a few. Using these methods, the gelatin is cross-linked by several types of chemical crosslinking agents such as, 1-ethyl-3-(3-dimethyl aminopropyl)-carbodiimide (EDC) and N-hydroxysuccinimide (NHS), glycidoxyproyltrimethoxysilane (GPTMS), glutaraldehyde, and genipin. The water-in-oil method is a commonly used laboratory technique but possesses many drawbacks including difficulties in scaling up to industrial scale and the use of oil and chemical crosslinkers results in toxicity issues requiring extensive removal of the residual oils and chemical crosslinkers.

One embodiment of the disclosure is directed to a method of preparing the plurality of microparticles of the disclosure, comprising: (a) mixing a cross-linkable protein solution and a cross-linker solution, where the cross-linkable protein solution comprises dissolving a cross-linkable protein comprising at least one RGD (Arg-Gly-Asp) motif or linked thereto (e.g., gelatin (e.g., recombinant gelatin, non-recombinant gelatin, in situ crosslinking), collagen (e.g., recombinant collagen, non-recombinant collagen), casein, tropoelastin, elastin, albumin, engineered protein thereof, and the like, or any combinations thereof; or non-recombinant gelatin, recombinant gelatin, non-recombinant collagen, recombinant collagen, any engineered protein thereto, engineered polymer comprising RGDs or linked thereto, and the like, or any combinations thereof) in a liquid (e.g., water, saline, PBS); and where the cross-linker solution comprises dissolving a cross-linker or an enzyme cross-linker (e.g., transglutaminase (e.g., natural transglutaminase, modified transglutaminase, recombinant transglutaminase, microbial transglutaminase (mTG), tissue transglutaminase (tTG), keratinocyte transglutaminase, epidermal transglutaminase, prostate transglutaminase, neuronal transglutaminase, human transglutaminase, Factor XIII, and the like, or any combinations thereof), oxidative enzyme (e.g., natural oxidative enzyme, modified oxidative enzyme, lysyl oxidase, tyrosinase, laccase, peroxidase, and the like, or any combinations thereof) in a liquid (e.g., water, saline, PBS), where the cross-linker is in an amount sufficient to crosslink the cross-linkable protein to form a cross-linked foam/block, non-foam cross-linked hydrogel, or fibers (such as in electrospinning). Another embodiment is directed to the cross-linker in an amount sufficient to convert the cross-linkable protein from soluble to insoluble at a temperature ranging from 10° C.-40° C. The method of preparing the plurality of microparticles of the disclosure further comprises: (b) forming a cross-linked foam/block, a non-foam cross-linked hydrogel, or fibers (such as in electro-spinning) comprising the mixed cross-linkable protein and the cross-linker of (a); (c) pulverizing the non-foam cross-linked hydrogel, fibers, or cross-linked foam of (b); (d) removing the cross-linker from the cross-linked formulation or product of (c) to form a cross-linker-free foam or hydrogel or fibers (e.g., essentially or substantially cross-linker-free); and (e) reducing in size: the formed cross-linked product of (d), the cross-linker-free product of (d), or combinations of the formed cross-linked foam of (d) and the cross-linker-free foam or hydrogel of (d), to form a plurality of particles and/or microparticles comprising size-reduced cross-linked foam or hydrogel of (b) and/or size-reduced cross-linker-free foam or hydrogel of (d). In some embodiments, the plurality of particles and/or microparticles comprising size-reduced cross-linked foam or hydrogel of (b) and/or size-reduced cross-linker-free foam or hydrogel of (d) can be sterilized by any appropriate method that does not substantially alter functionality, physico-chemical properties. stability, toxicity, or biological effects, including but not limited to: filtration, autoclaving (e.g., 110° C.-134° C.; 15 mins-40 mins; 5 psi-20 psi), irradiation (e.g., Ultraviolet (UV); gamma; electron beam (e-beam); X-rays). Some embodiments of sterilization include UV treatment under an exposure of 5 mins-720 mins (e.g., 100 mins, 150 mins, 200 mins, 250 mins) and UV wavelength of 10 nm-400 nm (e.g., 200 nm-270 nm). Additional embodiments include gamma irradiation of 10 kGy-50 kGy (e.g., 15 kGy, 20 kGy, 25 kGy, 30 kGy, 35 kGy, 40 kGy, 45 kGy). Vetten et al. disclose various sterilization techniques and parameters useful that can be applied herein and is incorporated by reference in its entirety (see, Nanomedicine. 10(7):1391-1399, 2014).

Some embodiments are directed to methods of preparing a plurality of microparticles as described here where the crosslinking occurs in vitro as a production-controlled step, in contrast to other formulations that are mixed at a point of care and injected, thereby allowing for crosslinking to occur in situ. In order to remove the transglutaminase crosslinker enzyme as described in the disclosed method, after the crosslinking reaction occurs, multiple repeated and extended washings are performed.

In another embodiment, the disclosed method of preparing a plurality of microparticles is directed to the cross-linkable protein solution, comprising: (i) adding a cross-linkable protein to a liquid (e.g., water, saline, PBS) at a temperature sufficient to dissolve the cross-linkable protein, such as a temperature greater than or equal 25° C. (e.g., 30° C., 37° C., 40° C., 45° C., 50° C.), where the cross-linkable protein is selected from, but not limited to, a protein comprising at least one RGD (Arg-Gly-Asp) motif (e.g., gelatin (e.g., non-recombinant gelatin, recombinant gelatin), collagen (e.g., non-recombinant collagen, recombinant collagen), casein, albumin, and any combinations thereof), at a temperature sufficient to dissolve the cross-linkable protein, essentially dissolve, or completely dissolve, such as but not limited to 40° C.-60° C., e.g., 50° C., while continuously stirring; and (ii) dissolving, essentially dissolving, or completely dissolving the cross-linkable protein in the liquid to form the cross-linkable protein solution.

Some embodiments are directed to fabricating foamed cross-linked gelatin microparticles (MPs) by a crosslinking reaction with a transglutaminase enzyme (e.g., microbial transglutaminase (mTG); recombinant transglutaminase; bacterial transglutaminase). Briefly, a transglutaminase (e.g., mTG) solution can be added to liquid state gelatin in a whipping machine or for mixing or stirring by any other means with or without gas or air (e.g., argon, carbon dioxide, helium, hydrogen, krypton, methane, neon, nitrogen, oxygen, ozone, water vapor, xenon, or any combinations thereof). In some embodiments, the method comprises forming a cross-linked foam by whipping the cross-linkable protein solution of (a) while adding the cross-linker solution of (a) at 37° C. to form the cross-linked foam of (b). Other embodiments are directed to the method of the disclosure comprising stirring or mixing the cross-linkable protein solution of (a) while adding the cross-linker solution of (a) at 37° C. without gas or air to form the non-foam cross-linked block of (b).

While mixing and foaming, the gelatin is crosslinked until a three-dimensional (3D) foam structure stabilizes. Afterward, the formulated foam is incubated at 45° C. and then chopped into large or gross slices or pieces (e.g., 0.05 cm-2 cm; 0.5 mm-20 mm). The chopped slices are washed several times at 50° C. for the removal of excess crosslinker or transglutaminase (e.g., mTG). After washing, the foamed gelatin is freeze-dried using, for example, a lyophilizer. For the creation of MPs, the dry crosslinked foamed gelatin is milled and sieved into microparticles in several size ranges (e.g., 0.1 μm-10 mm). The MPs can be sterilized by any means, including those described here, that does not negatively impact the structure, function, or performance of the microparticles, including, but not limited to, radiation.

In another embodiment of the disclosure, cross-linked gelatin microparticles (MPs) can be fabricated by a crosslinking reaction with a transglutaminase enzyme (e.g., microbial transglutaminase (mTG); recombinant transglutaminase; bacterial transglutaminase). Briefly, transglutaminase (e.g., mTG) solution is added to liquid state gelatin. The gelatin is stirred (with no foaming) until crosslinking into a stable three-dimensional (3D) structure, forming a crosslinked gelatin structure. Afterward, the formulated structure is incubated at 45° C. and then chopped into large or gross slices or pieces (e.g., 0.05 cm-2 cm; 0.5 mm-20 mm). The chopped slices are washed several times at 50° C. for the removal of excess crosslinker or transglutaminase (e.g., mTG). After washing, the crosslinked gelatin is then freeze-dried using, for example, a lyophilizer. For the creation of MPs, the dry crosslinked gelatin is milled and sieved into microparticles in several size ranges (e.g., 0.1 μm-10 mm). The MPs can be sterilized by any means, including those described here, that does not negatively impact the structure, function, or performance of the microparticles, including, but not limited to, radiation.

A further embodiment provides such method of preparing a plurality of microparticles directed to the cross-linker solution, comprising: (i) adding a cross-linker to a liquid (e.g., water, saline, PBS) at a temperature sufficient to dissolve the cross-linker, essentially dissolve, or completely dissolve, such as but not limited to room temperature, 15° C.-27° C., e.g., 25° C., while continuously stirring; and (ii) dissolving, essentially dissolving, or completely dissolving the cross-linker in the liquid to form the cross-linker solution. Other embodiments can be directed to such methods of preparing a plurality of microparticles, where the cross-linkable protein is cross-linked in the presence of, or when mixed with, the cross-linker of the disclosure. In a further embodiment, the cross-linker is an enzyme (e.g., transglutaminase, such as microbial transglutaminase) that when mixed with the cross-linkable protein, forms enzymatically cross-linked proteins, enzymatically cross-linked foams, or enzymatically cross-linked particles, or enzymatically cross-linked fibers. The cross-linked foam of (b) in methods of preparing a plurality of microparticles can be formed by: (b1) whipping the cross-linkable protein solution of (a) or (b2) mixing or stirring with or without gas or air (e.g., argon, carbon dioxide, helium, hydrogen, krypton, methane, neon, nitrogen, oxygen, ozone, water vapor, xenon, or combinations thereof), while adding the cross-linker solution of (a) at a temperature sufficient for the whipping, stirring to form the cross-linked foam of (b) or cross-linked block of (b), respectively, where, for example, the whipping or mixing or stirring occurs at a temperature of 30° C.-40° C. (e.g., 37° C.).

The removal of the cross-linker from the cross-linked protein, cross-linked foam, and/or microparticles or compositions comprising the same, in one embodiment, is beneficial from a safety and regulatory position, as well as cost. Accordingly, some embodiments can be directed to such methods of the disclosure where removing of (c), comprises: washing the cross-linked foam or block of (b), where the cross-linked foam or block of (b) is reduced in size as described here, where the washing occurs by agitating the pieces of cross-linked foam in a liquid (e.g., water, saline, PBS) at a temperature (e.g., 40° C.-60° C.; 45° C.-55° C., such as 50° C.) and time (e.g., 5 mins-1 hour; 10 mins-45 mins; 15 mins-30 mins) sufficient to remove or essentially remove the cross-linker from the cross-linked foam; and reducing in size by, for example, sieving the washed foam pieces to a desired size using an appropriate mesh sieve, such as but not limited to, a sieve with 35 mesh#-5000 mesh#(500 um to 2.5 μm), for example, a 0.5 mm mesh or 35 mesh# equivalent, sieve, thereby forming cross-linker-free foam pieces of the description comprising pieces of desirable sizes.

In one embodiment, such methods can provide a reducing in size of (d), comprising: cutting (e.g., dicing, chopping, meshing) the formed cross-linked foam or block of (b), the cross-linker-free foam or block of (c), or combinations of cross-linked foam or block of (b) and the cross-linker-free foam or block of (c). Other non-limiting examples of techniques, methods, or tools for reducing in size the cross-linked foam or block of (b) and/or the cross-linker-free foam or block of (c) include: cutting (e.g., dicing, chopping, meshing, sieving), using compression, lump breakers, pulverizers, mills (e.g., impact mills, flour mills, full-screen hammer mills, mega hammer mills, air classifying mills, jet mills, ball mills, pebble mills, rod mills); grinders (fine grinders, blade grinders), and the like, or combinations thereof. The reduction in size of such methods can occur to form a plurality of particles of 0.1 μm-10 mm (e.g., 0.2 μm-9 mm; 0.3 μm-8 mm; 0.4 μm-7 mm; 0.5 μm-7 mm; 1 μm-6 mm; 5 μm-5 mm; 10 μm-4 mm; 20 μm-1 mm; 40 μm-500 μm; 60 μm-200 μm; 90 μm-150 μm; 95 μm-100μ); greater than 1 μm (e.g., 2μ, 4μ, 6μ, 8μ, 12μ, 15μ, 25μ, 35μ, 45μ, 55μ, 65 μm, 75 μm, 85 μm, 95 μm, 105 μm, 115 μm, 125 μm, 135 μm, 145 μm, 150 μm, 200 μm, 300 μm, 400 μm, 500 μm, 1 mm, 3 mm, 5 mm, 7 mm, 9 mm); 10 mm or less (e.g., 8 mm, 6 mm, 4 mm, 2 mm, 900 μm, 800 μm, 700 μm, 600 μm, 550 μm, 450 μm, 350 μm, 250 μm, 175 μm, 165 μm, 155 μm, 140 μm, 130 μm, 120 μm, 100 μm, 90 μm, 80 μm, 70 μm, 60 μm, 50 μm, 40 μm, 30 μm, 20 μm, 10 μm, 5 μm, 3 μm, 1 μm). Additional embodiments provide such methods where the reducing of (d) results in the formed cross-linked foam of (b) or cross-linked foam pieces with a size of 0.5 mm-10 mm (e.g., 1 mm-8 mm; 2 mm-7 mm; 3 mm-6 mm; 4 mm-5 mm); 0.5 mm or greater (e.g., 1.5 mm, 2.5 mm, 3.5 mm, 4.5 mm, 5.5 mm, 6.5 mm, 7.5 mm, 8.5 mm, 9.5 mm); 10 mm or less (e.g., 9 mm, 8 mm, 7 mm, 6 mm, 5 mm, 4 mm, 3 mm, 2 mm, 1 mm).

In yet another embodiment, such methods of preparing a plurality of microparticles, comprises: (e) freezing the cross-linker-free foam or block of (c) or plurality of particles of (d); lyophilizing the frozen cross-linker-free foam or block of (e); and reducing in size the lyophilized cross-linker-free foam or block of (f) to form a plurality of cross-linked foam or block particles. Other embodiments of the methods of preparing a plurality of microparticles comprises: drying the cross-linker-free foam or block of (c) or plurality of particles of (d); and reducing in size the dried cross-linker-free foam or block of (c) to form a plurality of dried cross-linked foam or block particles. The plurality of cross-linked foam particles comprises a particle size of, for example, 0.1 μm-2000 μm (e.g., 0.2 μm-1499 μm; 0.4 μm-1450 μm; 0.5 μm-1425 μm; 0.6 μm-1400 μm; 0.7 μm-1350 μm; 0.8 μm-1300 μm; 0.9 μm-1250 μm; 1 μm-1200 μm; 2 μm-1150 μm; 3 μm-1100 μm; 4 μm-1050 μm; 5 μm-1000 μm; 6 μm-950 μm; 7 μm-900 μm; 8 μm-850 μm; 9 μm-800 μm; 10 μm-750 μm; 11 μm-700 μm; 12 μm-650 μm; 13 μm-600 μm; 14 μm-550 μm; 15 μm-500 μm; 16 μm-450 μm; 17 μm-400 μm; 18 μm-350 μm; 19 μm-300 μm; 20 μm-250 μm; 25 μm-200 μm; 30 μm-150 μm; 40 μm-100 μm; 60 μm-90 μm). Such methods comprise cross-linkable proteins selected from the group consisting of: gelatin, collagen, casein, albumin, tropoelastin, elastin and any combinations thereof; or non-recombinant gelatin, recombinant gelatin, non-recombinant collagen, recombinant collagen, any engineered protein thereof, engineered polymer comprising at least one RGD motif or linked thereto, and the like, or any combinations thereof. A further embodiment of such methods also comprises an enzyme cross-linker, where the enzyme cross-linker can be selected from transglutaminase or oxidative enzyme. Other embodiments of the disclosure provide for an enzyme cross-linker selected from the group consisting of: natural transglutaminase, modified transglutaminase, recombinant transglutaminase, microbial transglutaminase (mTG), tissue transglutaminase (tTG), keratinocyte transglutaminase, epidermal transglutaminase, prostate transglutaminase, neuronal transglutaminase, human transglutaminase, Factor XIII, and the like, or any combinations thereof. Some other embodiments can provide for an enzyme cross-linker selected from the group consisting of: natural oxidative enzyme, modified oxidative enzyme, lysyl oxidase, tyrosinase, laccase, peroxidase, and the like, or any combinations thereof.

Further embodiments provide for such methods, where the freezing of (e) occurs at a temperature sufficient for preparation of lyophilization, where the temperature comprises −18° C.-25° C. (e.g., −15° C. −23° C.; −10° C. −20° C.; −5° C.-15° C.; 0° C. −10° C.; −18° C. or greater (e.g., −16° C.; −14° C.; −12° C.; −8° C.; −6° C.; −4° C.; −2° C.; 2° C.; 4° C.; 6° C.; 8° C.; 10° C.; 12° C.; 14° C.; 16°; 18°; 20°; 22° C.; 24° C.); or 25° C. or less (e.g., 23°; 21° C.; 19°; 17°; 15° C.; 13° C.; 11° C.; 9° C.; 7° C.; 5° C.; 3° C.; 1° C.; −1° C.; −3° C.; −5° C.; −7° C.; −9° C.; −11° C.; −13° C.; −15° C.; −17° C.) for a time sufficient for preparation of lyophilization, where the time comprises: 2 hours-25 hours (e.g., 7 hours-24 hours; 9 hours-22 hours; 11 hours-20 hours; 13 hours-18 hours; 15 hours-16 hours); 5 hours or greater (e.g., 6 hours; 8 hours; 10 hours; 12 hours; 14 hours; 16 hours; 18 hours; 20 hours; 22 hours; 24 hours); or 25 hours or less (e.g., 23 hours; 21 hours; 19 hours; 17 hours; 15 hours; 13 hours; 11 hours; 9 hours; 7 hours; 5 hours).

Yet other embodiments can be directed to such methods, where the lyophilizing of (f) occurs at a temperature selected from: −50° C.±10° C. (e.g., −60° C.-−40° C.; −55° C.-−35° C.; −50°-−30° C.; −45°-−25°; −40° C. −20° C.; −35°-−30° C.; −60° C. or greater (e.g., −58° C.; −56° C.; −54° C.; −52° C.; −50° C.; −48° C.; −46° C.; −44° C.; −42° C.; −40° C.); −40° C. or less (e.g., −41° C.; −43° C.; −45° C.; −47° C.; −49° C.; −51° C.; −53° C.; −55° C.; −57° C.; −59° C.), at an atmosphere of: 0.01 mbar-0.1 mbar (e.g., 0.02 mbar-0.08 mbar; 0.04 mbar-0.06 mbar); 0.01 mbar or greater (e.g., 0.03 mbar; 0.05 mbar; 0.07 mbar; 0.09 mbar); or 0.1 mbar or less; 0.08 mbar; 0.06 mbar; 0.04 mbar; 0.02 mbar; 0.01 mbar); for a time of 24 hours-96 hours (e.g., 48 hours-95 hours; 50 hours-94 hours; 52 hours-92 hours; 54 hours-90 hours; 56 hours-88 hours; 58 hours-86 hours; 60 hours-84 hours; 62 hours-82 hours; 64 hours-80 hours; 66 hours-78 hours; 68 hours-76 hours; 70 hours-74 hours); 48 hours or greater (e.g., 49 hours; 51 hours; 53 hours; 55 hours; 57 hours; 59 hours; 61 hours; 63 hours; 65 hours; 67 hours; 69 hours; 71 hours; 73 hours; 75 hours; 77 hours; 79 hours; 81 hours; 83 hours; 85 hours; 87 hours; 89 hours); or 96 hours or less (e.g., 94 hours; 92 hours; 90 hours; 88 hours; 86 hours; 84 hours; 82 hours; 80 hours; 78 hours; 76 hours; 74 hours; 72 hours; 70 hours; 68 hours; 66 hours; 64 hours; 62 hours; 60 hours; 58 hours; 56 hours; 54 hours; 52 hours; 50 hours; 48 hours; 36 hours); where the temperature, the pressure, and the time are sufficient to result in a lyophilized frozen cross-linker-free foam of (c) or a lyophilized plurality of particles of (d), where a “lyophilized” product, such as but not limited to, a lyophilized cross-linker-free foam of (c) or a lyophilized plurality of particles of (d), as used here, is meant a product with a moisture content of 4% or less (e.g., 3.8%; 3.6%; 3.4%; 3.2%; 3%; 2.8%; 2.6%; 2.4%; 2.2%; 2%; 1.8%; 1.6%; 1.4%; 1.2%; 1%; 0.8%; 0.6%; 0.4%; 0.2%; 0.08%; 0.06%; 0.04%; 0.02%; 0%); 0% or greater (e.g., 0.01%; 0.03%; 0.05%; 0.07%; 0.09%; 1.1%; 1.3%; 1.5%; 1.7%; 1.9%; 2.1%; 2.3%; 2.5%; 2.7%; 2.9%; 3.1%; 3.3%; 3.5%; 3.7%; 3.9%); or 0%-4% (0.5%-3.5%; 0.7%-3.3%; 0.9%-3.1%; 1.1%-2.9%; 1.3%-2.7%; 1.5%-2.5%). The temperature, pressure, and time necessary to sufficiently lyophilize the cross-linker-free foam or plurality of particles are understood by those of ordinary skill in the art, and would not take undue experimentation to optimize these parameters.

In yet another embodiment, such methods of preparing a plurality of microparticles, comprises: (e) freezing the cross-linker-free foam or block of (c) or the plurality of microparticles of (d); and/or (f) drying the cross-linker-free foam or hydrogel block of (c) or (e) or the plurality of microparticles of (d). Some embodiments are directed to drying, including but not limited to, lyophilizing or freeze-drying, oven drying, and room temperature or ambient temperature drying. The method of preparing a plurality of microparticles, further comprising: (g) reducing in size the dried cross-linker-free foam or hydrogel block of (e) and/or (f) to form a plurality of cross-linked foam particles or non-foam cross-linked hydrogel particles. The plurality of cross-linked microparticles comprises a particle size of, for example, 0.1 μm-2000 μm (e.g., 0.2 μm-1499 μm; 0.4 μm-1450 μm; 0.5 μm-1425 μm; 0.6 μm-1400 μm; 0.7 μm-1350 μm; 0.8 μm-1300 μm; 0.9 μm-1250 μm; 1 μm-1200 μm; 2 μm-1150 μm; 3 μm-1100 μm; 4 μm-1050 μm; 5 μm-1000 μm; 6 μm-950 μm; 7 μm-900 μm; 8 μm-850 μm; 9 μm-800 μm; 10 μm-750 μm; 11 μm-700 μm; 12 μm-650 μm; 13 μm-600 μm; 14 μm-550 μm; 15 μm-500 μm; 16 μm-450 μm; 17 μm-400 μm; 18 μm-350 μm; 19 μm-300 μm; 20 μm-250 μm; 25 μm-200 μm; 30 μm-150 μm; 40 μm-100 μm; 60 μm-90 μm).

Another embodiment can be directed to such methods, where the reducing in size of (g), comprises: pulverizing the dried (e.g., lyophilized) cross-linker-free foam or hydrogel block of (e) to form the plurality of cross-linker-free foam particles; and separating by size the plurality of cross-linker-free foam or hydrogel particles of the disclosure. Such methods, where the plurality of cross-linker-free foam particles or hydrogel particles comprises a particle size of, for example, 0.1 μm-2000 μm, can comprise the reducing in size or separating by size of the plurality of cross-linker-free foam particles, which occurs by sieving the plurality of cross-linker-free foam particles sufficient to generate the plurality of cross-linked foam particles having different particle size ranges selected from a particle size or mean particle size of 0.1 μm-2000 μm, where the different particle size ranges comprise at least two different particle size ranges.

### Compositions

In some embodiments, the disclosure can be directed to a composition comprising (a) the plurality of microparticles as described here; and with or without (b) a carrier. The compositions of the disclosure comprises (a) a plurality of microparticles, where the plurality of microparticles comprises a cross-linked protein, where the cross-linked protein comprises at least one RGD (Arg-Gly-Asp) motif; where the plurality of microparticles is cross-linker-free; where the plurality of microparticles is all or independently water insoluble; and with or without (b) a carrier. Moreover, the compositions of the disclosure are injectable. Some embodiments are directed to compositions comprising the plurality of microparticles comprising at least two different particle sizes in a range of 0.1 μm-2000 μm (e.g., 5 μm-150 μm); or combinations thereof.

In another embodiment, compositions of the disclosure comprise: (a) a plurality of microparticles as described here, where the microparticles or plurality of microparticles comprise a cross-linked protein, where the protein of the cross-linked protein comprises at least one RGD (Arg-Gly-Asp) motif, where the plurality of microparticles is essentially or substantially cross-linker-free, where the plurality of microparticles is water insoluble; and optionally (b) a carrier. Such compositions comprising the plurality of microparticles of the disclosure comprises at least two different particle sizes in a range of 0.1 μm-2000 μm (e.g., 0.2 μm-1499 μm; 0.4 μm-1450 μm; 0.5 μm-1425 μm; 0.6 μm-1400 μm; 0.7 μm-1350 μm; 0.8 μm-1300 μm; 0.9 μm-1250 μm; 1 μm-1200 μm; 2 μm-1150 μm; 3 μm-1100 μm; 4 μm-1050 μm; 5 μm-1000 μm; 6 μm-950 μm; 7 μm-900 μm; 8 μm-850 μm; 9 μm-800 μm; 10 μm-750 μm; 11 μm-700 μm; 12 μm-650 μm; 13 μm-600 μm; 14 μm-550 μm; 15 μm-500 μm; 16 μm-450 μm; 17 μm-400 μm; 18 μm-350 μm; 19 μm-300 μm: 20 μm-250 μm; 25 μm-200 μm; 30 μm-150 μm; 40 μm-100 μm; 60 μm-90 μm) or the at least two different particle sizes comprise a mean particle size in a range of 0.1 μm-2000 μm (e.g., 0.2 μm-1499 μm; 0.4 μm-1450 μm; 0.5 μm-1425 μm; 0.6 μm-1400 μm; 0.7 μm-1350 μm; 0.8 μm-1300 μm; 0.9 μm-1250 μm; 1 μm-1200 μm; 2 μm-1150 μm; 3 μm-1100 μm; 4 μm-1050 μm; 5 μm-1000 μm; 6 μm-950 μm; 7 μm-900 μm; 8 μm-850 μm; 9 μm-800 μm; 10 μm-750 μm; 11 μm-700 μm; 12 μm-650 μm; 13 μm-600 μm; 14 μm-550 μm; 15 μm-500 μm; 16 μm-450 μm; 17 μm-400 μm; 18 μm-350 μm; 19 μm-300 μm; 20 μm-250 μm; 25 μm-200 μm; 30 μm-150 μm; 40 μm-100 μm; 60 μm-90 μm); and (b) a carrier.

Yet another embodiment of the disclosure provides such compositions as disclosed here, where the cross-linked protein is selected from the group consisting of: gelatin, collagen, elastin, tropoelastin, casein, albumin, any engineered proteins thereof, similar proteins thereof, and the like, or combinations thereof. In a further embodiment, the cross-linked protein can be selected from the group consisting of: non-recombinant gelatin, recombinant gelatin, non-recombinant collagen, recombinant collagen, engineered protein thereof, any engineered polymer comprising a RGD motif or linked thereto, and the like, or any combinations thereof. Other embodiments can be directed to a plurality of microparticles and such compositions described here comprising such plurality of microparticles, where the protein of the cross-linked protein comprises gelatin or collagen. In a further embodiment, the plurality of microparticles and described compositions comprising such plurality of microparticles are directed to proteins of the cross-linked protein that are comprised of gelatin.

The carrier, in some composition embodiments, can comprise a hydrogel. Some aspects of the embodiment provide a hydrogel carrier, where a “hydrogel” as used here in one embodiment means a gel or semi-solid hydrophilic polymer of at least 10% H2O. The carrier and/or lubricant can also be selected from the group consisting of, but not limited to: gelatin (e.g., crosslinked (2% w/v); non-crosslinked gelatin (0.25%-2% w/v)); or in situ crosslinked gelatin (0.1% w/v-10% w/v); collagen (e.g., crosslinked; non-crosslinked); alginate; carboxymethyl cellulose (CMC) (1%-3.5% w/v); poly(ethylene oxide) (PEO); poly(vinyl alcohol) (PVA); poly(propylene fumarate) (PPF); polyethylene glycol (PEG); glycosaminoglycan polymers such as hyaluronic acid (HA) (e.g., crosslinked and non-crosslinked HA (0.01%-10% w/v)); and the like, or any combinations thereof. The carrier can comprise a single carrier or a mixture of two or more carriers (e.g., a first carrier and a second carrier of the same different weight average molecular weights). Non-limiting examples of the carrier include glycosaminoglycan polymers (e.g., hyaluronic acid, crosslinked hyaluronic acid, keratan sulfate, chondroitin sulfate, and/or heparin), extracellular matrix protein polymers (e.g., gelatin, collagen, elastin, and/or fibronectin). Other embodiments are directed to compositions of the disclosure comprising a plurality of microparticles and a carrier, where the carrier is selected from the group consisting of: gelatin; collagen; alginate; glycosaminoglycan (GAG); polyethylene glycol (PEG); carboxymethyl cellulose; and combinations thereof. Some embodiments provide for compositions of the disclosure comprising a carrier selected from the group consisting of: uncrosslinked chondroitin sulfate polymers, uncrosslinked dermatan sulfate polymers, uncrosslinked keratan sulfate polymers, uncrosslinked heparan polymers, uncrosslinked heparan sulfate polymers, uncrosslinked hyaluronan polymers, uncrosslinked glycosaminoglycan polymers, uncrosslinked elastin and/or fibronectin, and any combinations thereof.

Further embodiments provided herein is an injectable composition comprising crosslinked hyaluronic acid carrier and plurality of microparticles, wherein the crosslinked hyaluronic acid has a crosslink density of about 3 mol % to about 40 mol %.

In some embodiments where there are at least two carriers, the first carrier may comprise hyaluronic acid with a weight average molecular weight of about 200 kDa to about 1 MDa, and optionally wherein the second carrier comprises hyaluronic acid with a weight average molecular weight of about 200 kDa to about 5 MDa. In some embodiments, the hyaluronic acid polymer may have a concentration of about 0.1% w/v to 10% w/v.

The average particle size of the protein microparticles in some embodiments involving the compositions described herein may be selected to suit the need of each application. For example, smaller average particle size may be desirable for treatment of fine lines and wrinkles, while larger average particle size may be more suitable for vocal fold augmentation or even large volume reconstruction (e.g., breast reconstruction).

Other embodiments are directed to compositions of the disclosure comprising: a plurality of microparticles described here and a carrier. Non-limiting examples of a carrier useful in embodiments of the disclosure is selected from the group consisting of: non-crosslinked gelatin; non-crosslinked collagen; non-crosslinked alginate; non-crosslinked hyaluronic acid; and combinations thereof. Whereas a non-active crosslinker as stored, can be added and reacted with a non-crosslinked carrier in situ, thereby maintaining the particles in place for injection. Another embodiment provides, for example, a non-crosslinked gelatin cross-linkable protein and active cross-linker enzyme that can crosslink in situ, thereby maintaining the particles in a hydrogel for a longer time in situ as compared to with the non-active cross-linker.

In one embodiment, the compositions of the disclosure comprising a plurality of microparticles that are cross-linker free, yet comprise cross-linked proteins, and a carrier, where the compositions have a concentration of the plurality of microparticles in the carrier of: 1 mg/ml or greater (e.g., 10 mg/ml; 20 mg/ml; 30 mg/ml; 40 mg/ml; 50 mg/ml; 60 mg/ml; 70 mg/ml; 80 mg/ml; 90 mg/ml; 100 mg/ml; 110 mg/ml; 120 mg/ml; 130 mg/ml; 140 mg/ml; 150 mg/ml; 200 mg/ml; 300 mg/ml); 300 mg/ml or less (e.g., 290 mg/ml; 280 mg/ml; 270 mg/ml; 260 mg/ml; 250 mg/ml; 240 mg/ml; 230 mg/ml; 220 mg/ml; 210 mg/ml; 200 mg/ml; 190 mg/ml; 180 mg/ml; 170 mg/ml; 160 mg/ml; 155 mg/ml; 145 mg/ml; 135 mg/ml; 125 mg/ml; 115 mg/ml; 105 mg/ml; 95 mg/ml; 85 mg/ml; 75 mg/ml; 65 mg/ml; 55 mg/ml; 45 mg/ml; 35 mg/ml; 25 mg/ml; 15 mg/ml; 5 mg/ml); or 1 mg/ml-300 mg/ml (e.g., 2 mg/ml-295 mg/ml; 4 mg/ml-285 mg/ml; 6 mg/ml-275 mg/ml; 8 mg/ml-265 mg/ml; 12 mg/ml-255 mg/ml; 14 mg/ml-245 mg/ml; 16 mg/ml-235 mg/ml; 18 mg/ml-225 mg/ml; 22 mg/ml-215 mg/ml; 24 mg/ml-205 mg/ml; 26 mg/ml-195 mg/ml; 28 mg/ml-185 mg/ml; 32 mg/ml-175 mg/ml; 34 mg/ml-165 mg/ml; 36 mg/ml-153 mg/ml; 38 mg/ml-143 mg/ml; 42 mg/ml-133 mg/ml; 52 mg/ml-123 mg/ml; 62 mg/ml-113 mg/ml; 72 mg/ml-103 mg/ml; 82 mg/ml-93 mg/ml).

In some embodiments involving the foam particles described here, the population of the foam particles can have an elastic modulus of at least about 0.5 kPa or greater (as measured at a 0.1 Hz-10 Hz frequency sweep).

Some embodiments provide the microparticle or plurality of microparticles described here, where at least about 40% (e.g., at least about 50%, at least about 60%, at least about 70%, or more) of the microparticle pores have an aspect ratio of about 1.0 to about 2.0.

In additional embodiments involving the particle described here, the pores of the particle have an average aspect ratio of about 1 to about 2.5.

Further embodiments provide microparticles or a plurality of microparticles described here, where the microparticles can be hydrated, for example, in an aqueous solution, including, but not limited to water, saline, a buffered solution, such as a phosphate buffered solution, or combinations thereof.

### Tissue Scaffolds

Another embodiment provides a tissue scaffold, comprising: a plurality of microparticles as described here, where the plurality of microparticles comprises cross-linked protein microparticles, where the plurality of microparticles comprises a protein of the cross-linked protein selected from, for example, gelatin; collagen; and combinations thereof, where the plurality of microparticles is water insoluble; and where the plurality of microparticles comprise a particle size of 1 μm-2000 μm (e.g., 5 μm-150 μm) or a mean particle size of 1 μm-1500 μm (e.g., 5 μm-150 μm). In some embodiments, the tissue scaffold further comprises a hydrogel carrier, where the hydrogel carrier is selected from, but not limited to, gelatin; collagen; alginate; hyaluronic acid; carboxymethyl cellulose; poly(ethylene oxide) (PEO); poly(vinyl alcohol) (PVA); poly(propylene fumarate) (PPF); polyethylene glycol (PEG), and the like, or any combinations thereof. Other embodiments are directed to such tissue scaffolds, comprising a dispersion of cross-linked protein microparticles, or a dispersion of the plurality of microparticles as described here, in a hydrogel carrier. Further embodiments provide for such a tissue scaffold, where the tissue scaffold is configured as a foam. In yet another embodiment, the tissue scaffold of the disclosure comprising the plurality of microparticles of cross-linked protein microparticles, where the plurality of microparticles is cross-linker-free and water insoluble, and the cross-linked protein microparticles or plurality of microparticles comprise at least two different or independent particle sizes. Yet other embodiments provide for such tissue scaffolds of the disclosure, where the tissue scaffold comprises or is configured in a three-dimensional shape. One embodiment is directed to the tissue scaffold having at least two different or independent particle sizes that comprise a particle size selected from: 1 μm-2000 μm (e.g., 5 μm-120 μm; 40 μm-100 μm; 60 μm-90 μm).

### Apparatus

Further embodiments of the disclosure provide an apparatus comprising the composition described here. In one embodiment, the apparatus of the disclosure comprises such compositions comprising: a plurality of microparticles, where the plurality of microparticles comprises a cross-linked protein, where the protein of the cross-linked protein comprises at least one RGD (Arg-Gly-Asp) motif, where the plurality of microparticles comprising cross-linked proteins or composition comprising the plurality of microparticles is cross-linker-free and water insoluble; and a carrier, such as a hydrogel, where the apparatus is a syringe, cartridge, or a vial. Other embodiments provide a syringe comprising: (a) a plurality of microparticles comprising cross-linked gelatin, where the plurality of microparticles is essentially or substantially cross-linker free and water insoluble; and (b) a hydrogel carrier, or compositions comprising the same, where the syringe and/or its contents therein, are sterilized, sterilizable, or configured for sterilization. Non-limiting examples of sterilization methods, techniques, or tools thereof include: steam sterilization (e.g., autoclave); flaming; heat sterilization (e.g., hot air ovens for dry heat sterilization; glass bead sterilizers); chemical sterilization (e.g., ethylene oxide gas sterilization, nitrogen dioxide sterilization, sterilization using glutaraldehyde and formaldehyde solution, hydrogen peroxide sterilization (e.g., liquid and vaporized), peracetic acid sterilization); radiation sterilization (e.g., electromagnetic radiation using ultraviolet (UV) light sterilization (e.g., UV-C or germicidal UV sterilization (e.g., far-UVC sterilization); gamma rays, X-ray; or irradiation by electron beams); broad-spectrum UV (including but not limited to, UV-A, UV-B, and UV-C wavelengths, or any combinations thereof); low-temperature sterilization (e.g., vaporized hydrogen peroxide, peracetic acid immersion, ozone)); and the like, or any combinations thereof. Further embodiments of the disclosure provide an apparatus, such as a syringe, where the syringe comprises a needle selected from 14 gauge (G) to 39 G (e.g., 18 gauge-30 gauge; 20 gauge-29 gauge; 22 gauge-27 gauge; 25 gauge-26 gauge; 27 gauge-30 gauge; 17 G; 18 G; 19 G; 20 G; 21 G; 22 G; 23 G; 24 G; 25 G; 26 G; 27 G; 28 G; 29 G; 30 G), where the lower the gauge (i.e., thicker needle), the easier it is to inject the plurality of microparticles or compositions of the disclosure; whereas, the higher the gauge (i.e., thinner needle), the less damaging to the dermis of a subject in need of the tissue scaffold, plurality of microparticles; or compositions comprising the plurality of microparticles. Some embodiments for dermatological applications can include, for example, a syringe apparatus that can be attached to or configured to attach to several different needles, such as 27 gauge-39 gauge needles. For instance, syringes with a 27 gauge needle with the said biomaterial that allow for injection of the particles and/or compositions of the disclosure while maintaining an injection force or load of 2 N-70 N (e.g., 3 N-60 N; 4N-50 N; 5 N-40 N; 6 N-30 N; 7 N-20 N); 70 N or less than 70 N (e.g., 65 N; 55 N; 45 N; 35 N; 25 N; 15 N; 5 N); 2 N or greater than 2 N (e.g., 3 N; 4 N; 5 N; 6 N; 7 N; 8 N; 9 N; 10 N; 11 N; 12 N; 13 N; 14 N; 15 N; 16 N; 17 N; 18 N; 19 N; 20 N; 21 N; 22 N; 23 N; 24 N; 25 N; 26 N; 27 N; 28 N; 29 N; 30 N; 40 N; 50 N; 60 N; 70 N) is encompassed by some methods of the disclosure. The tissue scaffold can be, in some embodiments, a porous, gelatin tissue scaffold. Other such embodiments provide a three-dimensional tissue scaffold. Further embodiments can be directed to an apparatus, such as a syringe, comprising a plurality of microparticles, a composition comprising the plurality of microparticles of the disclosure, or a tissue scaffold described here.

Further embodiments of the disclosure provide an apparatus, such as a syringe, a cartridge, a vial, or an additive manufacturing device (such as for biofabrication) comprising the microparticles of the disclosure, where the microparticles are placed into a plate or supportive hydrogel, tissue, or onto or into a subject's body.

In some embodiments of the disclosure, the compositions of any of the embodiments described here, provide an injectable composition that can be pre-loaded in an apparatus or delivery apparatus, such as a syringe. In some embodiments, the syringe is coupled to a tube via a handle so that the composition can be injected through the tube. This tube can further be coupled to an endoscope or cystoscope during a procedure. The needle can be a hollow needle that is attached to the tube. The tube can be positioned within and moveable within an outer sheath tube. The needle can be moveable between a retracted position within the outer sheath tube and an extended position in which the needle tip is outside the outer sheath tube to control injection of the compositions. In some embodiments, the outer sheath tube, with the needle and inner tube inside the outer sheath tube, is inserted into the channel of an endoscope. The delivery apparatus can include a handle that can be actuated by a user to move the inner tube distally relative to the outer tube sheath, thereby advancing the needle distally through the outer sheath tube toward an extended position in which the needle tip is exposed for injection of the compositions or plurality of microparticles as described here into a tissue or region of interest.

In other embodiments for small volume bulking applications, the composition or plurality of microparticles can be injected with a 14 gauge-39 gauge needle using an average extrusion force of no more than about 30 N. Examples of small volume bulking applications include, but are not limited to a dermal filler for skin tissue (e.g., treatment of facial skin tissue having a facial line, wrinkle, or a scar to be filled), bulking of urethra (e.g., treatment for stress-urinary incontinence), bulking of cervical tissue (e.g., treatment for cervical insufficiency), and bulking of vocal fold (e.g., correction of vocal fold paralysis or other causes of vocal fold insufficiency).

Uses and Methods of Treatment

Yet a further embodiment provides a use of the plurality of microparticles, the composition comprising the plurality of microparticles, the tissue scaffold, the apparatus comprising the plurality of microparticles and/or the composition comprising the plurality of microparticles, for any one or more of: body contouring, tissue engineering, regenerative medicine, and aesthetic dermatology, where some embodiments further provide for body contouring selected from the group consisting of: soft tissue reconstruction, volume restoration, breast augmentation, biostimulation, and the like, or any combinations thereof. Another use embodiment of the disclosure comprises biostimulation, as used here, that is selected from the group consisting of: fibroblast stimulation, collagen production stimulation, neo-collagenesis (i.e., process of making new collagen), tissue regrowth, inducing angiogenesis, providing a tissue scaffold, and the like, or any combinations thereof. Yet a further use embodiment of the disclosure relates to the composition and/or the plurality of microparticles configured in the apparatus described here, where the apparatus is, for example, a syringe, a cartridge, or a vial.

A method of the disclosure provides for a method of treating a subject (animal, including human) in need of body contouring as described here, comprising administering the composition of a plurality of microparticles and a carrier, at a site of the subject in need of body contouring. Such a method comprises administering by, for example, injecting the composition of a plurality of microparticles and a carrier, at a site of the subject in need of body contouring. Another embodiment of the disclosure provides for such method of treating a subject in need of body contouring, where administering comprises: stimulating fibroblasts; stimulating collagen production; inducing neo-collagenesis; inducing tissue regrowth; inducing angiogenesis; providing a tissue scaffold; and the like, or any combinations thereof.

In some embodiments of the disclosure, the plurality of microparticles suspended in a hydrogel carrier to form a composition, can be injected at a site of a subject via a sterile syringe containing the composition, where the subject is in need of therapeutic and/or aesthetic applications. The compositions or formulations described here can be injected into the subcutaneous layer (aka subcutis, hypodermis), soft tissue, and mammalian glands as needed. This technique can be utilized in conjunction with others in order to visualize the injection placement, for example, ultrasounds and X-rays. Furthermore, injecting a tissue scaffold into a subject, using minimally invasive procedures, minimizes: the risk of infection from performing open surgery, costs associated with surgery, and/or potential for medical malpractice since exposure of the body cavity is minimized. Also, the methods of treating a subject described here by injecting the plurality of microparticles suspended in a hydrogel carrier as a composition also reduces recovery time and pain as compared to typical surgery that requires a large excision or opening greater than the size of a syringe and/or needle used here.

Some embodiments are directed to administration types of the composition or formulation described her, where the compositions or formulations are administered into the subcutaneous layer (also known as subcutis or hypodermis) of the skin. Skin can include facial skin, buttocks skin, or any soft tissue. Compositions and formulations described here also include administration to the mammary gland or into fat tissues for breast reconstruction procedures in a subject. Pre-clinical data demonstrated gelatin microparticles of the disclosure with non-crosslinked gelatin carrier that were injected into the subcutaneous (SC) layer of the skin in a rat model and a pig model, as well as into the mammary gland in the pig model. See, e.g., EXAMPLE 2.

In some embodiments, a plurality of microparticles of the disclosure or a composition comprising a plurality of microparticles as a scaffold can be used to provide immediate physical and mechanical stabilization of a tissue defect or provide skin lifting/expansion through the biomechanical strength of the scaffold, which can be an implant. The implant of the disclosure can be used as a transitory scaffold for soft tissue support and repair to reinforce deficiencies where weakness or voids exist that require the addition of material to obtain the desired surgical outcome. After implantation, the implant and/or ingrown native tissue resulting from the implant, can maintain at least 10% volume of the time zero implant volume (i.e., 100% time 0 volume) after 1 month, 3 months, or 6 months' time. The implant can act as a filler for, e.g., body contouring. reconstruction, breast augmentation, that does not immediately degrade, and is replaced with tissue stimulated by the implant (e.g., stimulating fibroblasts and/or collagen production; inducing neo-collagenesis; inducing tissue regrowth; providing a tissue scaffold; or the like, or any combinations thereof). Since the implant can act as a biostimulator, stimulated cells or tissue remain for 3 months-6 months in a subject, in a volume of, for example, 10%-100% (e.g., 20%-50%) volume of the initial implant. New cells or tissue can be induced by the implant and replaces the microparticle implant.

Another embodiment provides for the plurality of microparticles and/or composition of the disclosure that act as a biostimulant and tissue scaffold. For example, when the plurality of microparticles and/or composition is administered, fibroblasts and collagen production can be stimulated, neo-collagenesis and/or tissue regrowth can be induced, and/or a tissue scaffold utilized, all within the boundaries of a safe, effective, and inexpensive tissue scaffold and/or biostimulant for use in therapeutic, aesthetic dermatology, and reconstructive procedures or surgery.

As illustrated in EXAMPLE 16, some embodiments of the disclosure provide a use of the composition comprising a plurality of microparticles or the plurality of microparticles, where the composition or the plurality of microparticles functions as a microcarrier for living cells, for either in vitro or in vivo applications. In vitro cultures of cells comprising. for example, foam particles (FPs) described here can be utilized for the production of proteins, biomaterials for research, medical purposes such as micro-organs for drug development, microstructures for tissue engineering, or as agents for enhancement of cell-based therapies. In some embodiments, these cells can proliferate and be maintained in vast numbers, for example, in a continuous manner. However, this can be difficult to achieve in standard two-dimensional cell culture methods (i.e., superficial culture in plastic plates, flasks, etc.) and the microparticles (e.g., FPs) of the disclosure, for example, can act as microcarriers allowing for three-dimensional, suspended culture, optimally utilizing culture volume and media, and enabling single batch as well as continuous culture processes. Furthermore, such microcarriers can provide support for cells used in vivo in cell-based therapies, e.g., cells injected into tissues, enhancing cell survival in vivo. The microparticles of the disclosure (e.g., FPs) can be also used for cells differentiation using stem cells or satellite cells with optimal conditions for the desired cells line differentiation, such as environmental conditions, seeding concentration and choice of medium.

Another embodiment provides for ex vivo tissue engineering, for example, 3-dimensional (3D) scaffolding to support cell growth and promote formation of tissue-like micro-organs for implantation or for use in drug screening or for protein manufacturing.

In some embodiments, the compositions or plurality of microparticles of the disclosure can be used for in vitro tissue or cell culturing, where upon contacting cells (e.g., mammalian cells) with the compositions or plurality of microparticles, the cells proliferate and as such express specific proteins, and the cells can be expanded to express more proteins. Some embodiments are directed to mammalian cells, which require an RGD rich scaffold to grow or expand. Non-limiting examples of cells include: fibroblasts, epithelial, Chinese hamster ovary (CHO), NS0 and Sp2/0 murine myeloma cell, HEK293 cells, Human diploid (HeLa) cells, baby hamster kidney (BHK21) cells, and the like, which express proteins selected from the group consisting of: structural extracellular matrix (ECM) components such as collagen, elastin, gelatin, hormones, monoclonal antibodies, enzymes, FC-fusion protein, cytokines and growth factors, clotting factors, respectively. For example, the compositions or plurality of microparticles of the disclosure can be used as a microcarrier or scaffold for cell attachment, growth, expansion, or combinations thereof, where the cells can be any commonly known and used mammalian, adherent cells, such as but not limited to: fibroblasts, epithelial, Chinese hamster ovary (CHO), NS0 and Sp2/0 murine myeloma cell, HEK293 cells, Human diploid (HeLa) cells, baby hamster kidney (BHK21) cells, cardiomyocytes, induced pluripotent stem cells, and the like. In some aspects, fibroblasts, cardiomyocytes, and induced pluripotent stem cells are commonly used cells that are representative of other cell types used for expression and research on small organs.

Additional embodiments are directed to uses of the compositions or plurality of microparticles described here, for protein purification by in vitro tissue or cell culturing. In some embodiments, protein purification can be accomplished by collecting the expressed proteins, filtering the proteins in the culture medium, where filtering or separating the proteins that are water soluble from the microparticles that are water insoluble, occurs by, for example, filtration or centrifugation, for collection.

Some embodiments are directed to a method of producing a protein (e.g., cell-free), comprising: growing or culturing a plurality of protein-producing or-expressing cells in a cell culture comprising a plurality of microparticles or a composition comprising a plurality of microparticles described here and culture medium, under conditions sufficient to culture the cells and inducing protein expression or synthesis. In some embodiments, the cells are mammalian cells (e.g., fibroblasts, epithelial cells, Chinese hamster ovary (CHO), NS0 and Sp2/0 murine myeloma cell, HEK293 cells, Human diploid (HeLa) cells, baby hamster kidney (BHK21) cells which can be used to produce a protein selected from the group of: structural ECM components such as collagen, elastin, gelatin, hormones, monoclonal antibodies, enzymes. FC-fusion protein, cytokines and growth factors consisting of: Hormones; Choriogonadotropin alfa, Follitropin alfa, Follitropin beta, Luteinizing hormone, Osteogenic protein-1. Thyrotropin alfa, Clotting factors, Factor VIII, Factor IX, Insulin, Somatropin, collagen, _antibodies; Adalimumab, Alemtuzumab, Bevacizumab, Brentuximab, Denosumab, Golimumab, Ibritumomab tiuxetan, Ipilimumab, Obinutuzumab, Omalizumab, Pertuzumab, Rituximab, Siltuximab, Tocilizumab, Trastuzumab, Vedolizumab, Ado-trastuzumabemtansine, Ustekinumab. Enzymes; Agalsidase beta, Alglucosidase alfa, Alteplase, Elosulfasc, GalNAc 4-sulfatase, Human DNase, Hyaluronidase, Imiglucerase, Laronidase, Tenecteplase, growth factors; and cytokines; Darbepoetin alfa, Interferon beta-1a, Epoetin alfa, Epoetin beta, Epoetin theta, and the like.

Embodiments of the disclosure are also directed to a method of culturing any of the aforementioned cells (e.g., mammalian, adherent) on a microcarrier, where a microcarrier is a plurality of microparticles described here, having a dry particle size of 5 μm-2000 μm (e.g., 99 μm-700 μm).

In some embodiments, the cells are adherent, mammalian cells, such as human fibroblasts, epithelial cells, Chinese hamster ovary (CHO), NS0 and Sp2/0 murine myeloma cell, HEK293 cells, Human diploid (HeLa) cells, baby hamster kidney (BHK21) cells, and any of the aforementioned cells which are common and representative of the type of cells useful for in vitro cell culturing for protein expression or purification.

Some embodiments of the disclosure provide for a method of producing a protein, e.g., a cell-free protein, comprising: growing a plurality of protein-producing cells in a cell culture comprising the plurality of microparticles of the disclosure and culture medium, where the growing occurs under conditions that induce protein synthesis, thereby producing a cell-free protein. Non-limiting examples of protein-producing cells include: fibroblasts for collagen production, epithelial cells, Chinese hamster ovary (CHO) for production of monoclonal antibodies such as: Adalimumab, Alemtuzumab, Bevacizumab, Brentuximab, Denosumab, Golimumab, Ibritumomab tiuxetan, Ipilimumab, Obinutuzumab, Omalizumab, Pertuzumab, Rituximab, Siltuximab, Tocilizumab, Trastuzumab, Vedolizumab, Ado-trastuzumabemtansine, Ustekinumab or enzymes production such as: Agalsidase beta, Alglucosidase alfa, Alteplase, Elosulfasc, GalNAc 4-sulfatase, Human DNase, Hyaluronidase, Imiglucerase, Laronidase, Tenecteplase, or hormone production such as: Choriogonadotropin alfa, Follitropin alfa, Follitropin beta. Luteinizing hormone, Osteogenic protein-1, Thyrotropin alfa, Clotting factors, Factor VIII, Factor IX, Insulin, Somatropin; NS0 and Sp2/0 murine myeloma cells for producing monoclonal antibodies such as: Belimumab, Natalizumab, Ofatumumab, Palivizumab, Ramucirumab, Abciximab, Basiliximab, Canakinumab, Cetuximab, Infliximab; and HEK293 cells, Human diploid (HeLa) cells, baby hamster kidney (BHK21) cells for the production of Clotting factors such as Factor VIIa or Factor VIII. In some embodiments, the methods of producing a cell-free, or essentially cell-free, protein described here, produces a protein or cell-free protein selected from the group consisting of: collagen; a hormone; a monoclonal antibody; an enzyme; a growth factor; a cytokine; and combinations thereof.

In some embodiments, a method of producing a differentiated cell or differentiated cells, comprises: growing a plurality of cells, including but not limited to induced pluripotent stem cells. dermal stem cells, epidermal stem cells, and the like. The plurality of cells are grown in a cell culture or cell culture medium comprising a plurality of microparticles or composition comprising the plurality of microparticles of the disclosure (i.e., a cross-linked protein comprising at least one RGD motif, where the plurality of microparticles does not comprise of, does not substantially comprise of cross-linker, or is cross-linker-free, or essentially cross-linker-free), wherein the cells are grown under conditions sufficient to induce cell differentiation, thereby producing differentiated cells. For example, the aforementioned plurality of cells can be differentiated into functional cells, such as functional cardiomyocytes.

Additional embodiments of the disclosure are directed to a method of culturing cells (e.g., mammalian, adherent) on a microcarrier, where a microcarrier comprises a plurality of microparticles described here, having a dry particle size of 5 μm-2000 μm. In some embodiments, the cells are adherent, mammalian cells suitable for differentiation, such as induced pluripotent stem cells (iPS), embryonic stem cells, hematopoietic stem cell, mesenchymal stem cell, satellite cells, and any of the aforementioned cells.

All terms used herein are intended to have their ordinary meaning in the art unless otherwise provided. All concentrations are in terms of percentage by weight of the specified component relative to the entire weight of the topical composition, unless otherwise defined.

As used herein, “a” or “an” shall mean one or more. As used herein when used in conjunction with the word “comprising,” the words “a” or “an” mean one or more than one. As used herein “another” means at least a second or more.

As used herein, all ranges of numeric values include the endpoints and all possible values disclosed between the disclosed values. The exact values of all half-integral numeric values are also contemplated as specifically disclosed and as limits for all subsets of the disclosed range. For example, a range of from 0.1% to 3% specifically discloses a percentage of 0.1%, 1%, 1.5%, 2.0%, 2.5%, and 3%. Additionally, a range of 0.1 to 3% includes subsets of the original range including from 0.5% to 2.5%, from 1% to 3%, from 0.1% to 2.5%, etc. It will be understood that the sum of all weight % of individual components will not exceed 100%.

By “consist essentially” it is meant that the ingredients include only the listed components along with the normal impurities present in commercial materials and with any other additives present at levels which do not affect the operation of the invention as described in the disclosed embodiments, for instance at levels less than 5% by weight or less than 1% or even 0.5% by weight.

### EXAMPLES

The following examples illustrate specific aspects of the instant description. The examples should not be construed as limiting, as the example merely provides specific understanding and practice of the embodiments and its various aspects.

For example, the examples here describe the preparation of the enzymatically (mTG) crosslinked gelatin foam microparticles which form a tissue scaffold in essence, describe rheological properties of the composition of the disclosure in the context of injectability, and demonstrate the safety and effectiveness of the microparticles and compositions of the disclosure as an injectable dermal filler, showing low inflammation and significant neo-collagenesis in animal model experiments.

### Example 1

### Preparation of Crosslinked Gelatin foam Microparticles

Microparticles of Crosslinked Gelatin Foam were Prepared as Follows:
1) Gelatin powder was gradually added to water at 50° C. under continuous stirring, until completely dissolved.
2) Separately, microbial transglutaminase (mTG) was gradually added to water at 25° C. under continuous stirring, until completely dissolved.
3) Dissolved gelatin solution was whipped into a foam at ˜37° C., using a whipping machine to aerate, or agitated or stirred, for example, with gas or air (e.g., argon, carbon dioxide, helium, hydrogen, krypton, methane, neon, nitrogen, oxygen, ozone, water vapor, xenon).
4) Dissolved mTG solution was gradually added into the gelatin solution, during stirring, for example, without gas or air, which continued until a crosslinked gelatin confluent hydrogel block was formed.
5) Foam or hydrogel block was diced or cut into pieces (e.g., 5 mm-20 mm (i.e., 2 cm)).
6) Diced hydrogel block or foam was washed twice, by agitating in water at 50° C. and filtering until mTG enzyme was removed or washed away, or the majority of enzyme was removed to form a crosslinker-free diced hydrogel or foam.
7) The washed, diced crosslinked gelatin foam or hydrogel block was frozen overnight (e.g. 2 hours-25 hours) on a tray at −18° C., and then lyophilized for 48 hours, 0.04 mbar-0.05 mbar.
8) The lyophilized foam or hydrogel block was pulverized either using a jet mill or blade grinder, and separated into particle size groups (e.g., 0.1 μm-2000 μm) by passing the powder through sieves (e.g., 35 US Mesh#-5000 US Mesh#; 2.5 μm-500 μm).

Gelatin was crosslinked with different microbial transglutaminase concentrations to generate a stable structure. The stable structure was chopped, milled, and sieved to several size ranges to form the microparticles, and washed to remove the transglutaminase as mentioned in the production process. The microparticles were diluted to different concentrations in a carrier or lubricant before injection. The microparticles and carrier were injected as a confluent homogenous gel-like fluid, with no presence of air (which is composed primarily of nitrogen and oxygen, and can also include small amounts of, for example, carbon dioxide, hydrogen, helium, argon, neon, etc.). Crosslinked gelatin foam microparticles of desired size range (e.g., 0.1 μm-2000 μm) were dispersed in a liquid carrier of choice (e.g., gelatin; hyaluronic acid; carboxymethyl cellulose; water) (see, e.g., TABLES 1-3), or mixed with dry powder of the carrier/lubricant component, filled into syringes, and sterilized by autoclave or radiation.

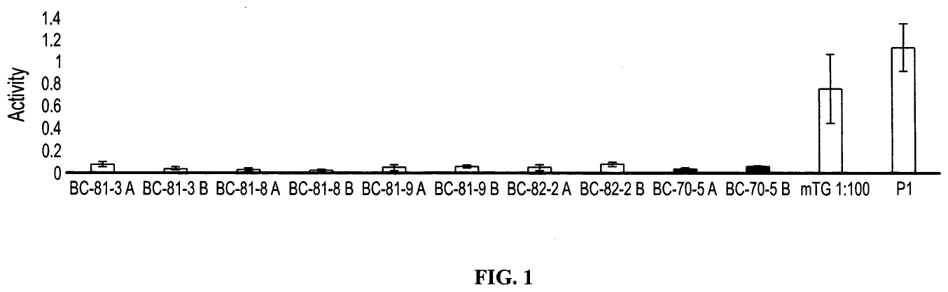

The amount of mTG crosslinker in the microparticles was measured using a mTG activity assay and SDS PAGE. The concentrations tested showed values of mTG activity and mTG protein lower than that of the positive control, demonstrating that the microparticles were essentially or substantially cross-linker free as the term is used herein. See, FIG. 1.

### Example 2

### Histopathological Evaluation of Crosslinked Gelatin Foam Microparticle Formulation

The acute and sub-chronic reaction to a subcutaneously injected formulation of crosslinked gelatin foam microparticles in a rat skin model was performed to evaluate safety, tolerability, and performance for tissue augmentation and skin remodeling. Parameters evaluated were external skin reactions as well as cell and tissue responses to the implanted crosslinked gelatin foam microparticle formulation.

For this experiment, a formulation of the disclosure was tested, which was composed of 125 mg of lyophilized gelatin foam microparticles, sterilized by radiation (10 Kilo Gray), and suspended in 1.2 ml sterile saline. The dry gelatin foam microparticles were mixed with saline 3 hours prior to injection. Preparation of the microparticles is described in more detail in EXAMPLE 1.

The gelatin foam microparticle formulation was implanted by injection into the subcutaneous tissue of three rats. Each rat was implanted at one to two sites with 0.3 ml of the formulation at each site. One site was injected with 0.3 ml of a competitive product, Radiesse™ (Merz Aesthetics; a collagen stimulator composed of calcium hydroxyapatite microspheres in aqueous gel carrier), which was used a positive control. Implantation sites were collected for histopathological evaluation by Hematoxylin & Eosin (HE) and Masson's trichrome (MT) staining at day 7 and day 30.

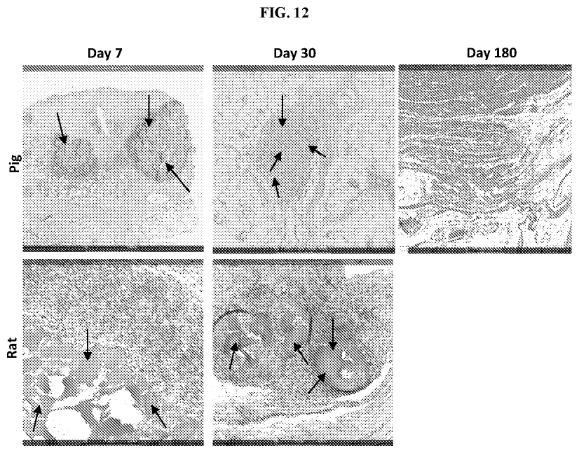

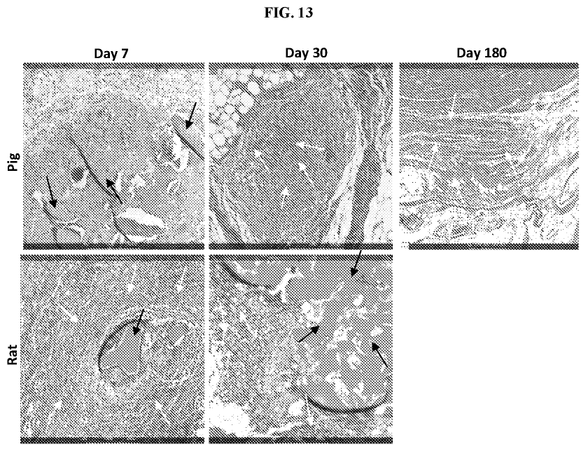

Histopathological evaluation was based on a semi-quantitative scoring method, and evaluated by an independent pathologist, in a “blinded” fashion. The evaluation of implant tolerability and performance consisted of parameters of local tissue response at the site of implantation, presence of necrosis, cavity formation, type of cell infiltration, presence of foreign body response, amount of new collagen fibers (neo-collagenesis), and material absorption. Each parameter was scored on a scale of 0-4 (where each number of the scale represented: 0—no change, 1—minimal, 2—mild, 3—moderate, 4—severe). See, FIGS. 12-13.

A histopathological evaluation of the injectability, safety, tolerability and performance of gelatin foam microparticle formulations of the disclosure for tissue augmentation and skin remodeling was performed.

The following formulations were prepared with various amounts of crosslinked gelatin foam microparticles per milliliter of product and various carrier hydrogels.

TABLE 1
In vivo Tested Formulations.

| | | | | Particles | | | | |
|---|---|---|---|---|---|---|---|---|
| | Carrier | | | | | Weight | Sites | |
| | | Concen- | Free/ | Dry/ | Size | (mg)/1 | (syringe | # of |
| # | Type | tration % | Xlink | Suspension | (μm) | mL | quantity) | Syringes |
| 1 | CMC | 2.5% | Free | Dry | 60-99 | 120 | 1 mL/ | 8 |
| | | | | | | | site × | |
| | | | | | | | 3 sites | |
| 2 | HA | 1% | Free | Suspension | 60-99 | 60 | 1 mL/ | 8 |
| | (3Molar) | | | | | | site × | |
| | | | | | | | 3 sites | |
| 3 | Gelatin | 0.5% | Free | Dry | 60-99 | 120 | 1 mL/ | 8 |
| | | | | | | | site × | |
| | | | | | | | 3 sites | |
| 4 | Gelatin | 2% | Xlinked | Dry | 60-99 | 60 | 1 mL/ | 12 |
| | | | | | | | site × | |
| | | | | | | | 3 sites | |
| 5 | Gelatin | 0.5% | Free | Dry | 60-99 | 60 | 1 mL/ | 8 |
| | | | | | | | site × | |
| | | | | | | | 3 sites | |
| 6 | Gelatin | 0.5% | Free | Dry | 60-99 | 30 | 1 mL/ | 8 |
| | | | | | | | site × | |
| | | | | | | | 3 sites | |
| 7 | HA | 1% | Free | Suspension | 60-99 | 30 | 1 mL/ | 8 |
| | (3Molar) | | | | | | site × | |
| | | | | | | | 3 sites | |
| 8 | Neg. | | | | | | 1 mL/ | |
| | Control | | | | | | site × | |
| | (saline) | | | | | | 2 sites | |

All of the formulations (#1-8) of TABLE 1 were injected successfully to each injection site: 1 ml per 3 cm×3 cm square in a pig belly. Carboxymethyl cellulose (CMC); hyaluronic acid (HA);

The pig skin was examined for macroscopic adverse events for one week and the injection sites were observed to lack any adverse reactions.

### Results:

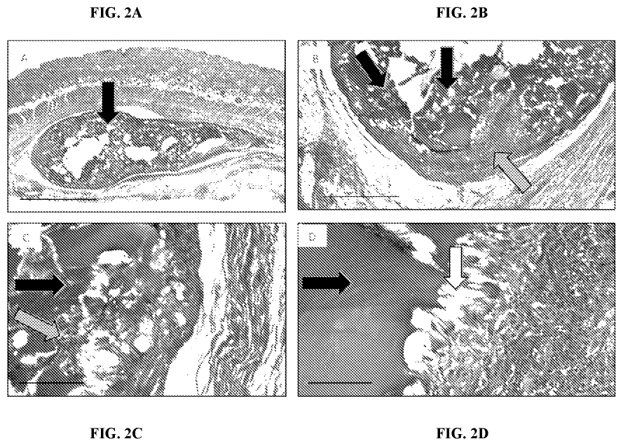

The disclosed gelatin foam microparticle formulation implants collected from two sites on day 7, showed a foreign body response graded 1 and neo-collagenesis graded 1-2. Implants from three sites were collected on day 30, which showed neo-collagenesis grade 2 with foreign body response graded 1-2, and some absorption of the implant was noticeable. Also observed, was an abundance of fibroblasts connecting with the implant or composition comprising a plurality of microparticles described here and penetrating the implant or composition comprising a plurality of microparticles. No necrosis, cavity formation, nor edema was present, at any site and time point, proving good tissue tolerability (FIG. 2A-FIG. 2D).

In comparison, a similar amount of the positive control, RadiesseTM (Merz Aesthetics), was implanted subcutaneously and collected on day 30 for histopathological evaluation. In the implanted site, neo-collagenesis was graded 0-1, and predominant foreign body response was graded 3. Whereas the gelatin foam microparticle formulation of the disclosure was graded 2 for neo-collagenesis and for predominant foreign body response, graded 1-2. The Radiesse™ implants permitted cell migration around the particles but did not allow infiltration into the particle bulk as seen with gelatin foam microparticles.

In summary, the gelatin foam microparticle formulation of the disclosure was found to be safe. The implant was observed to be highly tolerable with no negative effect on tissues such as muscles, blood vessels, nerves and epidermis. The implant promotes skin regeneration by stimulating neo-collagenesis, which is superior to the positive control competitive product.

### Example 3

### Injectability Characterization of Gelatin Foam Microparticle Formulation

The gelatin foam or hydrogel microparticle formulation of the disclosure was developed, in one embodiment, for aesthetic dermatology, and reconstructive surgery, to provide an optimal scaffold support for fibroblast stimulation and tissue regrowth. A product that solves the tremendous need for a safe and injectable bio-stimulant, with an immediate clinical outcome, while lifting the skin as similarly produced using dermal fillers and with a long-lasting result, is desired.

Injectability is considered to be the ability of a product that is successfully administered by a syringe and appropriate needle. Injectability of the gelatin foam microparticle formulation of the disclosure was assessed using the Lloyd compression system (LLOYD Instruments). This method was developed for the characterization of an adhesive 3D foam structure according to ASTM F2900-11 Standard guide and characterization of hydrogels used in regenerative medicine. This analytical method provided mechanical data of the force needed to inject the material through a syringe. Needle size and syringe brand and size affect the force.

The purpose of this study was to assess different, and optimal formulations for administration of the gelatin foam microparticle formulation of the disclosure. The challenges in developing such a product included generating a uniform cohesive paste that could support the gelatin foam particles of the disclosure to maintain their 3D structure in the target tissue and to prevent early clearance, such as 3 months to 2 years. An ideal formulation would be stable during a reasonable period of storage time in refrigeration (e.g., 4° C.) or room temperature (e.g., 20° C.-25° C.).

Formulation is referred to as the specific combination of gelatin foam microparticle size, particle to carrier ratio, carrier type, and carrier concentration.

### Results

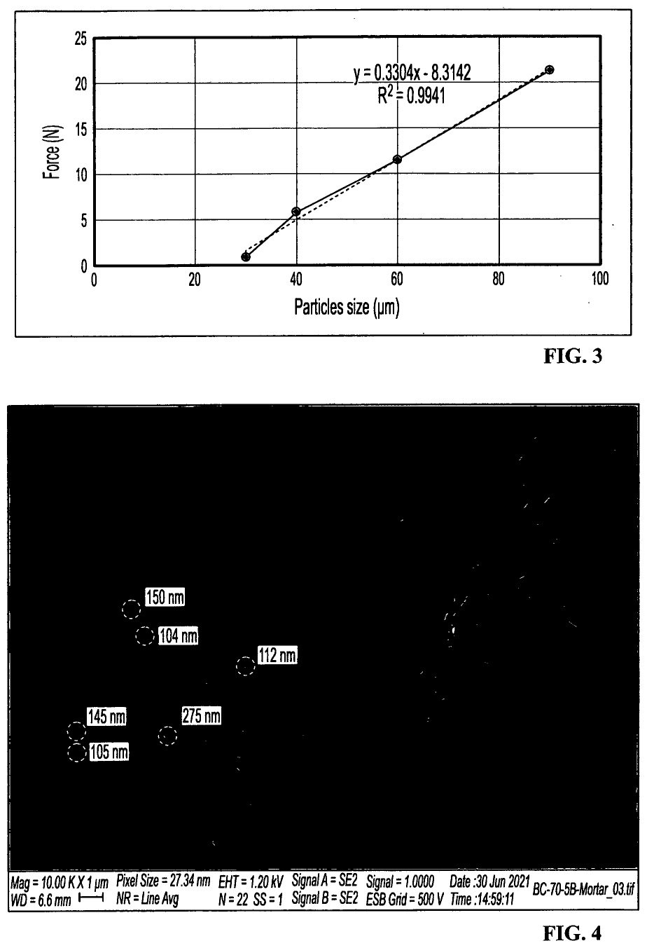

The effect of particle size on injection force was tested. Different size particles were suspended in carboxymethyl cellulose (CMC) carrier. Specifically, 120 mg of each particle size (e.g., 30 μm-100 μm) was suspended in 1 ml of 1% CMC. Injection force was measured using 1 ml syringes and 27 gauge needles. A linear correlation (dotted line) was observed between particle size and the force measured (solid line) (FIG. 3).

### Formulations

Dozens of different formulations were initially tested. Once a prepared formulation texture was identified as visually smooth and coherent, a syringe was prepared and its injectability tested. Accordingly, optimization of each of the formulations was performed in a step-by-step manner. The study results presented here used particles sizes 50 μm-100 μm (e.g., 60 μm-90 μm), vet additional formulations containing smaller particle sizes less than 60 μm (e.g., 30 μm, 40 μm) were also prepared.

TABLE 2
Formulations as an Injected Medical Product or Implant.

| | Carrier | | | Particles | | | |
|---|---|---|---|---|---|---|---|
| | | Concentration | Free/ | Dry/ | Size | | |
| # | Type | % | xlink | suspension | (μm) | Weight (mg) | Sterilization |
| 1 | CMC | 2.5% | Free | Dry | 60-90 | 120 mg/1 ml | Autoclave |
| 2 | HA | 1% | Free | Suspension | 60-90 | 70 mg/1 ml | Autoclave |
| | (3M) | | | | | | |
| 3 | Gelatin | 2% | Free | Dry | 60-90 | 110 mg/1 ml | Autoclave |

Stability after storage at 2° C.-8° C. was tested. Three time points were tested for formulation #1 in TABLE 2, namely time points of 1 day, 3-4 days, and 7 days. Prior to force tests, syringes were equilibrated to room temperature. As can be seen in TABLE 3, there was no significant change in injection force, after the tested time points of 1 day, 3-4 days, and 7 days of refrigerated storage.

TABLE 3
Injection Force After Refrigerated Storage of Formulation #1.

| | Replicate | Aging/ | Particle | Syringe | Needle | Load |
|---|---|---|---|---|---|---|
| Sample | # | Stability | Size | Type | Gauge | (N) |
| CMC 2.5% + | 1 | 1 day | 60 μm | 1 mL | 30 | 39 |
| 60 μm | 2 | 1 day | | | | 37 |
| CMC 2.5% + | 1 | 4 days | | | | 42 |
| 60 μm | 2 | 3 days | | | | 42 |
| CMC 2.5% + | 1 | 1 week | | | | 36 |
| 60 μm | 2 | 1 week | | | | 40 |

Gelatin foam microparticle formulations with various particle sizes and carrier hydrogels were prepared. The particles were milled to particle sizes of 30 μm, 40 μm, 60 μm, and 90 μm. Injectability was found to be affected by particle size in a linear correlation of particle size to injection force needed. Data were obtained for two different needle sizes: 30 G and 27 G, which are known to be suitable for minimally invasive dermatology application (FIG. 3).

### Example 4

### Morphological Shape of Gelatin Foam Microparticle Formulations in Different Size Ranges

The morphological structure of a gelatin foam microparticle (MP) was evaluated using High Resolution Scanning Electron Microscopy (HR-SEM) and bright field microscopy. Morphological parameters such as the shape, size and size distribution of MPs, and porosity and surface texture of the MPs were investigated.

A small quantity of MPs sample was placed in a 1.5 ml microcentrifuge flip-cap tube, for transportation. Samples were prepared for high resolution scanning electron microscopy (HR-SEM) (Technion; “Soft Material Electron Microscopy” unit). Specifically, double-sided adhesive carbo-tape pieces were adhered to designated metallic molds, onto which the sample MPs were spread and adhered evenly.

Analysis: several measurements were done on images, using the HR-SEM software. All other analyses are principally qualitative, as visual assessment of the images and graphical representation. See, FIGS. 4-6, FIG. 7A-7B, FIG. 8.

As seen in the HR-SEM images, particles in several size ranges were prepared. Particles having a particle size of greater than (or down to) 0.1 μm were imaged. See, FIG. 4; 104 nm, 105 nm, 112 nm, 145 nm, 150 nm, 275 nm. MPs having a particle size of 60 μm-99 μm were observed. See,

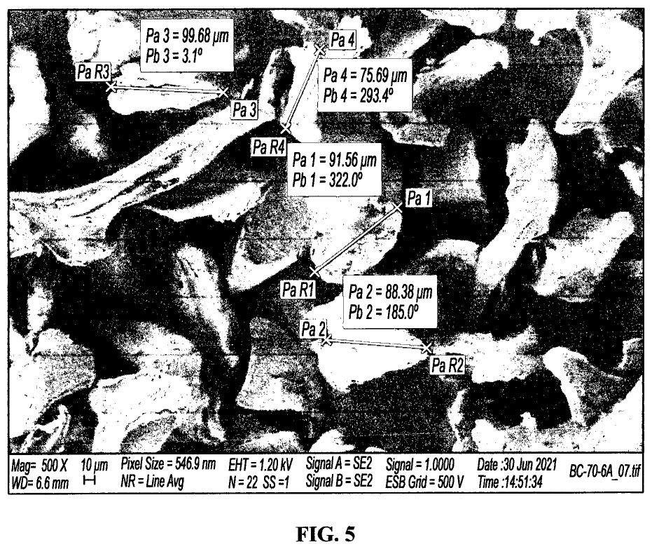

FIG. 5; 75.69 μm; 88.38 μm; 91.56 μm; 99.68 μm. The size range was controlled and adjusted during the milling and sieving production steps of the particles.

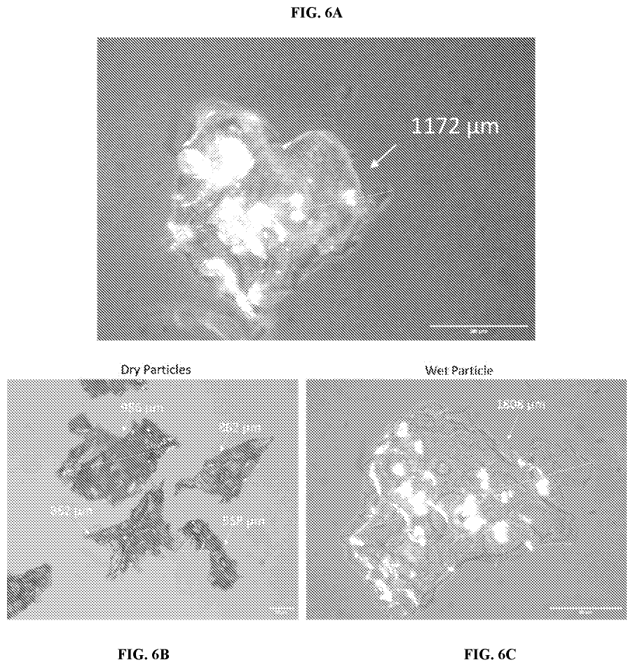

Large particles up to 2000 μm in size were observed using bright field microscopy. The particles were hydrated before imaging. Crosslinked gelatin was milled and sieved to a size range of 99 μm-710 μm. The resultant particles' swelling factor (wet/dry) was 1.65. Once wet, the particles' swell and size increased by a factor of 1.65 from their initial size, up to about 2000 μm. See, FIG. 6 which shows a hydrated gelatin particle of 1172 μm.

### Example 5

### Particle Size Determination

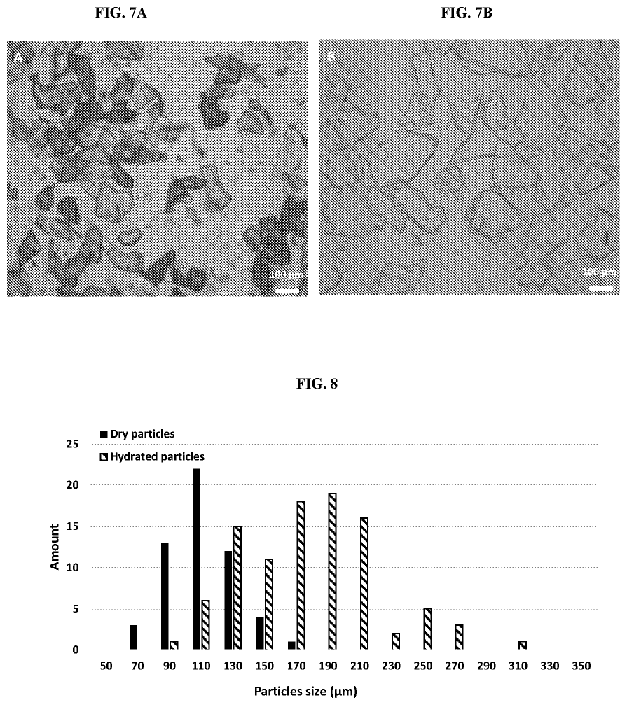

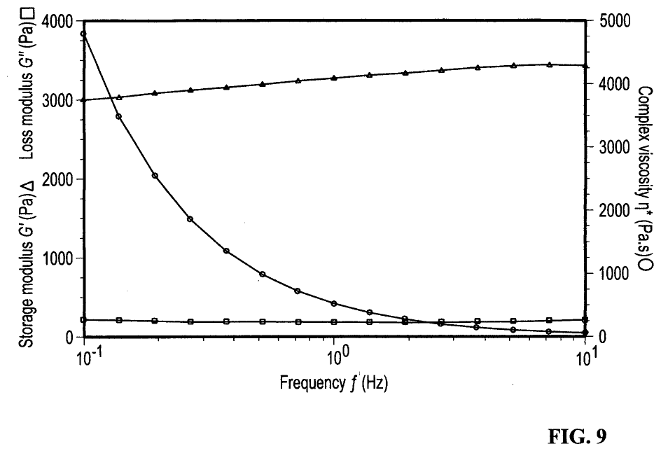

Lyophilized microparticles of crosslinked gelatin were dispersed in phosphate buffered saline (PBS) for 24 h at room temperature (RT). Hydrated microparticles were visualized under the light microscope and compared to the dry particles. The size distribution was manually evaluated by ImageJ software. See, FIG. 7A, FIG. 7B, FIG. 8.

TABLE 4
Summary of Exemplary Microparticle Size Ranges and Average.

| Size (μm) | Dry particles (n = 55) | Wet particles (n = 97) |
|---|---|---|
| Minimum | 60 | 85 |
| Maximum | 155 | 303 |
| Average (std) | 101 ± 20 | 167 ± 42 |

### Example 6

### Mechanical Properties of the Gelatin Foam Microparticle Formulations

The mechanical properties of the gelatin foam microparticle formulation were prepared and tested after mixing and storage for 1 hour at room temperature (RT) and at 6° C.

Wet and dry formulations of gelatin foam microparticles (MPs) (i.e., 60 μm-99 μm, 120 mg/ml) in 0.5%, 0.75% or 1% of non-crosslinked gelatin carrier were prepared and introduced in a 1 ml syringe. The formulation's mechanical properties were measured using an AG-R2 Rheometer with a frequency sweep test in a range of 0.1 Hz-10 Hz. See, FIG. 9. These different oscillation frequencies correspond to different levels of shear force that are being applied on the samples. Measurement of gel stiffness and thus its ability to resist deformation under applied pressure can give an indication of how the formulation is extruded through an injection needle or cannula, or how the formulation is subjected to movements of the facial musculature and overlying skin after.

The dry particle formulation resulted in a lower modulus than that of the wet particle formulation. This can be due to the difference between the formulations: the dry particle formulation was sterilized before mixing with liquid while the wet particle formulations were sterilized after mixing with a liquid. Moreover, the percentage of the non-crosslinked gelatin carrier was lower in the dry particle formulation.

TABLE 5
Mechanical Properties of Gelatin Microparticles in Non-Crosslinked
Gelatin Carrier at Room Temperature (RT) and 6° C.

| | | | | | |
|---|---|---|---|---|---|
| | | Storage | Loss | | Complex |
| Gelatin % | Storage | modulus | modulus | | viscosity |
| (w/v) | Temperature | (G′), [Pa] | (G″), [Pa] | tan δ | (n*), [Pa · s] |

Wet Formulation

| | | | | | |
|---|---|---|---|---|---|
| 0.75 | RT | 3300 ± 300 | 200 ± 10 | 0.059 ± 0.01 | 530 ± 40 |
| 1 | RT | 3400 ± 200 | 200 ± 20 | 0.058 ± 0.02 | 550 ± 40 |
| 0.75 | 6° C. | 3300 ± 100 | 189 ± 4 | 0.056 ± 0.01 | 530 ± 20 |
| 1 | 6° C. | 3800 ± 200 | 219 ± 2 | 0.058 ± 0.03 | 600 ± 30 |

Dry Formulation

| | | | | | |
|---|---|---|---|---|---|
| 0.5 | RT | 1458 | 103 | 0.07 | 232 |

### Example 7

### Size Characterization of Foam Gelatin Microparticles

Samples from different batches of foam particles made with different amounts of microbial transglutaminase (mTG) per 20-gram gelatin were taken for measurements in the Mastersizer 3000, (at the ITI Faculty of Biotechnology and Food Engineering, Technion, Haifa, IL). Samples were dispersed in deuterium-depleted water (DDW) or in 96% ethanol immediately before measurement or dispersed in water for 24 hours before measurement.

TABLE 6
Average Diameter Values of Foam Particles
Measured Using a Mastersizer 3000.

| Sample | | Dx | Dx | Dx | Dx |
|---|---|---|---|---|---|
| Name | Treatment | (10) | (50) | (90) | (99) |
| BC-81- | DDW, instant | 65 ± 1 | 137 ± 3 | 230 ± 5 | 305 ± 6 |
| 9 | DDW-24 h | 38 ± 1 | 133 ± 1 | 241 ± 2 | 337 ± 2 |
| | 96% Ethanol, | 50 ± 5 | 79 ± 1 | 117 ± 2 | 142 ± 2 |
| | instant | | | | |
| BC-82- | DDW, instant | 53 ± 14 | 129 ± 2 | 225 ± 3 | 304 ± 6 |
| 0 | DDW-24 h | 61 ± 3 | 137 ± 0.5 | 235 ± 3 | 320 ± 10 |
| | 96% Ethanol, | 51.9 ± 0.4 | 82 ± 1 | 123 ± 2 | 154 ± 5 |
| | instant | | | | |

Measurements were performed in 5 replicates (n=5).

### Results:

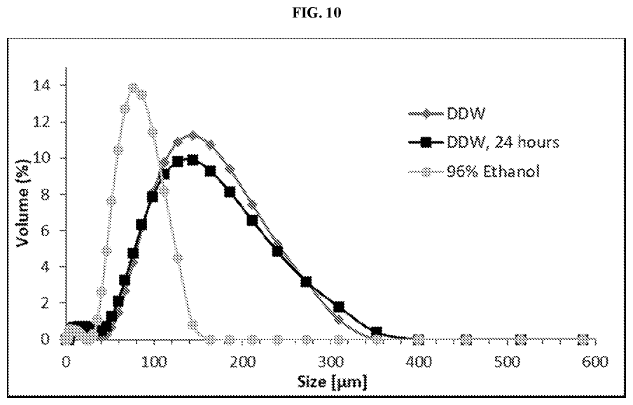

FIG. 10 displays the size distribution of non-hydrated foam MPs (BC 81-9) and hydrated foam MP. Foam MPs were dispersed in deuterium-depleted water (DDW) (either instantly (DDW) or for 24 hours (DDW, 24 hours)), and in 96% ethanol. The dispersion of the particles in 96% ethanol was the non-hydrated state of the particles, which indicated the dry particle size after sieving.

When the particles were dispersed in 96% ethanol, a narrower size distribution was observed, with a size range of 50 μm-150 μm, and Dₓ (50) of approximately 80 μm. Foam MPs were sieved to a size range of 60 μm-99 μm, resulting in dry particles having a size Dₓ (50) of 80 μm was approximately in the middle the expected sieving range.

When the particles were dispersed in water (DDW), a wider size distribution was observed. This was due to water uptake of the gelatin foam MPs, which caused swelling of the particles, and resulted in a shift in the size distribution plot. No significant difference was found between Dx (50) of particles hydrated in water instantly or after 24 hours of hydration, which indicated that foam MPs were fully hydrated soon after the dispersion in water.

The resulting ratio between the Dₓ (50) of the hydrated particles (DDW-24 hrs) to the non-hydrated particles (96% ethanol) was 1.67.

### Example 8

### Injectability of Foam Gelatin Microparticles in Different Saline Dilutions

220 mg foam microparticles with 10 mg of non-crosslinked gelatin used as a carrier were mixed with 1.5 ml, 2 ml, and 3 ml of saline in a 2.5 ml syringe in a syringe to syringe (STS) manner for 30 seconds.

Injectability was measured 10 min after mixing using a Lloyd's mechanical testing instrument with a 27-gauge (27 G) needle.

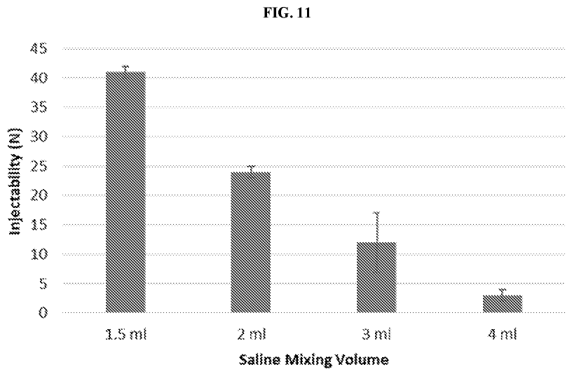

As displayed in TABLE 7 and in FIG. 11 the saline mixing volume affects the injectability values of the crosslinked gelatin foam MPs formulation. When the mixing volume was increased from 1.5 ml to 4 ml. the injectability decreased from 41 N to 3 N. respectively. This ability to adjust the injectability of the formulation could be an advantage when injecting the formulation in different locations and volumes depend on the tissue resistance.

### Example 9

### Sterility of Foam Gelatin Microparticles Formulations

The sterility of the gelatin foam MPs was evaluated using endotoxin and bioburden tests after sterilization using E-beam radiation of 12 kGy (Sor-Van, IL).

For evaluating endotoxin levels, 20 mg of foam gelatin MPs were dispersed in 5 ml endotoxin-free water with 4 U or 8 U collagenase and incubated at 37° C. under 150 rpm shaking overnight until full degradation of the foam MPs. The endotoxin values were quantified using EndoZyme™ II assay.

For evaluation of and quantification of bacterial levels or microbial contamination in water, raw materials, or finished products for safety purposes of a manufactured product, a bioburden test was performed. In compliance with ISO 11737-1, a bioburden test was performed externally at Miloda laboratories (SOP 200.04.01). A sample (0.1 g) was placed in 1 ml Buffered Sodium Chloride-Peptone (BSCP)+0.1% Tween. Extraction was performed by hand mixing for 60 sec, then 1 ml of the extraction was plated on Tryptic Soy Agar (TSA) plate and incubated at 30° C.-35° C. An amount of microorganisms that grew on the plate was counted after 72 hours. Afterward, the petri dishes were transferred to 25° C. for another 72 hours and then the number of yeast and molds were counted as colony forming units (CFU).

TABLE 8
Endotoxins and Bioburden of Foam
Gelation MPs After Sterilization.

| | Endotoxin | EU/ | |
|---|---|---|---|
| Sample (batch number) | [EU/mg] | Device | Bioburden |
| BC-83-1 & BC-83-2 | 0.0399 | 8.782 | <1 CFU/gram |
| BC-83-3 & BC-83-4 | 0.0466 | 10.255 | <1 CFU/gram |
| BC-83-5 & BC-83-6 | 0.0126 | 2.761 | <1 CFU/gram |

As seen in TABLE 8, the endotoxin level (endotoxin unit (EU)) of the foam particle samples was between 0.0126 EU/mg to 0.0466 EU/mg. When calculating the EU per device (of 220 mg of foam particles (FPs)), the EU value was up to 10.2, which is under the acceptable EU value of 20 EU per device demonstrating the sterility of the formulation and validation of the sterility method using E-beam radiation. This was also demonstrated by a bioburden test with colony forming unit (CFU)/gram (g) smaller than or less than 1.

### Example 10

### Water Insolubility Testing of Microparticles (MPs)

Microparticles of the disclosure were placed in a well (6 plate well) with 5 ml saline and incubated at 55° C., while on a shaker at 100 rpm. Water insolubility of the MPs was assessed at time zero (before incubation), 1 hour, and 4 days for visual assessment. No distinction was visually observed over the time course.

In another study, microparticles of the disclosure were placed in a filter that was pre-dried at 60° C. overnight. The filter was weighted with 50 mg-55 mg particles and placed in 2 ml Eppendorf tube. Water was added inside the filter to make sure that the particles were covered and the filter mesh was in contact with the water (˜2.5 ml). The filter and Eppendorf tube were covered with aluminum foil, sealed with tape and placed for incubation at 60° C. After 1 h or 48 h, the samples were washed and dried to measure weight loss. The samples were washed with 3 ml-4 ml water each, (300 μl-400 μl in 10 rounds) and placed at 60° C. overnight to dry. The filter with the FPs sample was weighted after the dryness process. The study was performed in triplicate. The weight ratios of dry FPs before incubation or soaking in water and after incubation in water at 60° C. for 1 h and 48 h were 1.006 and 1.005, respectively. Thus, the material dry mass remained unchanged when incubated in water for 1 hour or 48 hours, even when the water was warm (60° C.). Hence, FPs are crosslinked and not water soluble.

### Example 11

### Animal Implantation Safety Study

In a pig implantation safety study, different types of the formulation were injected into the subcutaneous tissue (also known as, subcutis, hypodermis) of the pig. The injection sites were analyzed up to 180 days post-injection (BC010 study in swine model). Microparticles (MPs)+carrier or lubricant formulations tested showed good acute and sub-chronic tolerability and was determined as safe. Fibroblast recruitment was shown even in the early time point of day 7, continued by a later process of neo-collagen production seen at day 30. Collagen stimulation was demonstrated, and with the support of local angiogenesis (new capillaries formation), lead to the generation of new, vivid collagenous tissue.

In a rat implantation study, MPs and carrier or lubricant formulations were injected into the subcutaneous tissue (also known as, subcutis, hypodermis) of the rat. The study showed high safety and tolerability of the injected formulation in different dosages up to 2 ml per injection site (in a rat model that is an extreme 100-fold overdosing) with no adverse events, edema, or necrosis up to 30 days post-injection. Collagen stimulation was demonstrated, and with the support of local angiogenesis (formation of new capillaries), lead to the generation of new collagenous tissue.

In a rat implantation study, FPs with carrier dry particles were mixed with a different hydrating liquid: saline, phosphate buffered saline (PBS), or water for injection (WFI). For example, 110 mg FPs and 5 mg non-crosslinked gelatin were mixed with 1 ml saline or WFI. The study showed high safety and tolerability of the injected formulation in different liquids, with no adverse events, edema, or necrosis up to 30 days post-injection. Collagen stimulation was demonstrated, and with the support of local angiogenesis (formation of new capillaries), lead to the generation of new collagenous tissue.

In a rat implantation study, FPs dry particles were mixed with different carriers: (a) 120 mg FPs were mixed with 0.5 ml saline and with 0.5 ml crosslinked Hyaluronic Acid (HA; is 3000 KD; 10 mg/ml; 0.05 BDDE/1 mg HA); (b) 120 mg FPs were mixed with a hygroscopic dry powder of 5 mg non-crosslinked gelatin (see, e.g., U.S. Pat. Nos. 10,596,194 and 11,331,412 regarding the particles) and dry powder of 12.5 enzyme units of microbial transglutaminase (mTG) mixed with 1 ml saline. Both formulations (a) and (b) showed high safety and tolerability with no adverse events, edema, or necrosis up to 30 days post-injection. Collagen stimulation was observed (only around the FPs and not around the hyaluronic acid), and lead to the generation of new collagenous tissue indicating that crosslinked gelatin microparticles FPs were critical to cell ingrowth and remodeling.

In a rat implantation study, dry FPs were fabricated by crosslinking the gelatin with different concentrations of mTG. 120 mg FPs of various crosslinking mTG formulations were mixed with 1 ml saline. All formulations showed high safety and tolerability with no adverse events, edema, or necrosis up to 30 days post-injection. Collagen stimulation was demonstrated, and with the support of local angiogenesis (formation of new capillaries), lead to the generation of new collagenous tissue.

### Example 12

### Evaluation of In Vivo Implanted Formulations

In a pig implantation study (BC010), the injected MPs and carrier formulations were observed in the implantation sites at day 7 with remnants seen at a 1-month time point. At 180 days, the MPs and carrier formulations were fully degraded with no remnants seen. In a rat study (PCR007), the injected MPs and carrier formulations were present in the implantation sites 1-month post-injection.

For the pig experiment, a formulation of the disclosure was tested, which was composed of 30 mg-120 mg of lyophilized gelatin foam microparticles in different carriers to a final volume of 1 ml, sterilized by autoclave. Preparation of the microparticles was described in more detail in EXAMPLE 1.

For the rat experiment, a formulation of the disclosure was tested, which was composed of 220 mg of lyophilized gelatin foam microparticles mixed with 10 mg of carrier powder, sterilized by radiation (10 Kilo Gray), and suspended in 2 ml sterile saline. The dry gelatin foam microparticles were mixed with 2 ml saline immediately prior to injection. Preparation of the microparticles is described in more detail in EXAMPLE 1.

The gelatin foam microparticle formulation was implanted by injection into the subcutaneous tissue of 2 pigs and 18 rats. Each rat was implanted at one to four sites with 0.3 ml to 2 ml of the formulation at each site. The arrows show the implanted composition of the disclosure. Implantation sites were collected for histopathological evaluation by Hematoxylin & Eosin (HE) and Masson's trichrome (MT) staining at day 7 and day 30 in the rat model and at day 7, day 30, and day 180 in the pig model. Implants were stained with H&E (Hematoxylin and Eosin which stains cell nuclei a purplish blue, and the extracellular matrix and cytoplasm pink) and MT, Mason Trichrome (produce red keratin, muscle fibers and implant, blue collagen and bone, light red or pink cytoplasm, and dark brown to black cell nuclei) in pig and rat skin at 7-, 30-, and 180-days post-implantation (H&E-pig Day 7 and Day 30, Rat Day 7 and Day 30, and MT-pig Day 180). See, FIG. 12.

The microparticle composition or formulation described here can be classified as biodegradable, which possesses an advantage in risk mitigation. Other commercially available products, such as calcium hydroxyapatite (CaHA) or poly-L-lactic acid (PLA), showed that degradation rates can cause numerous adverse events and complications. Treatment with CaHA had the highest complication rate with the most common adverse events of nodule and granuloma formation in the injected tissue. The CaHA-CMC (calcium hydroxyapatite-carboxymethylcellulose) implants permitted cell migration around the particles but did not allow infiltration into the particle bulk as seen with the gelatin microparticles.

In a rat study, the injected microparticle formulations were present in the implantation sites 1-month post-injection.

### Example 13

### Evaluation of Biostimulation Process

Based on pig and rat implantation studies as described here, the biostimulation process of the material was observed as early as 7 days post-injection. This was shown by an ongoing collagenesis process (grade 2) and the formation of new collagen fibers in the injected area. Representative histologic photographs of implants stained with H&E (Hematoxylin and Eosin which stains cell nuclei a purplish blue, and the extracellular matrix and cytoplasm pink) and Mason Trichrome (produce red keratin, muscle fibers and implant, blue collagen and bone, light red or pink cytoplasm, and dark brown to black cell nuclei) at 7-, 30-, and 180-days post-implantation (H&E-pig Day 7 and Mason Trichrome pig Day 30 and Day 180, Rat Day 7 and Day 30). The implanted formulation (black arrows) of new collagen fibers stained in blue (white arrows). See, FIG. 13.

### Example 14

### mTG Residues in Foam Particles (FPs) measured by SDS-PAGE

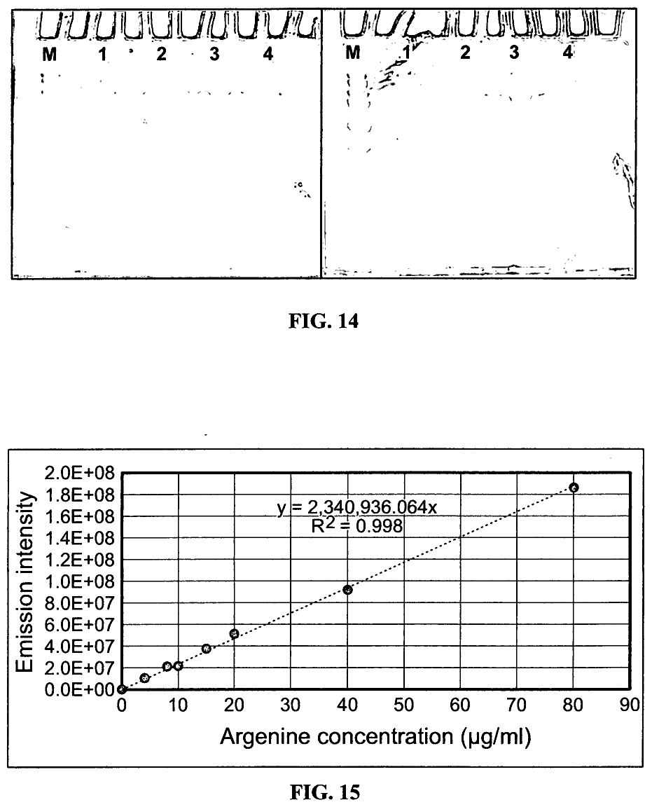

This study was to analyze by SDS-PAGE a qualitative measurement of microbial transglutaminase (mTG) enzyme residues in FPs product. The appearance of a mTG band in FPs suspension indicated the presence of the enzyme. The study was performed in duplicate. See, FIG. 14.

Test controls of mTG (1), gelatin (2), and collagenase (3) showed results in the expected protein pattern and size. See, FIG. 14.

The FPs result (4) showed no evidence or trace of mTG enzyme in the suspension. This suggested that the FPs contained none or a very minute amount of mTG if at all and mTG enzyme was undetectable as measured by this method.

### Example 15

### RGD Quantification

A fluorometric assay was used to quantify the amount of RGD (Arginine-Glycine-Aspartate) motifs on the surface of crosslinked gelatin microparticles and raw materials, through the amino groups of arginine. The reaction between amino groups of arginine with 9,10-phenanthrenequinone produced a fluorescent compound. The reaction typically occurs at a high pH followed by acidification that produces a fluorescent compound or molecule.

Samples and standards were separately mixed with a 9,10-phenanthrenequinone reagent in a high pH environment and incubated at 60° C., 100 rpm for 3 hours. Then, the mixture was mixed with HCl and incubated at room temperature (RT) for 1 hour to obtain the fluorescent molecule. The fluorescence intensity was measured with an excitation wavelength of 312 nm and emission of 395 nm. Blank control without RGD was prepared with deionized water. Samples were tested in triplicates.

### Results:

The fluorescence emission of Arginine was measured in different concentrations and shown in the calibration curve of arginine of FIG. 15.

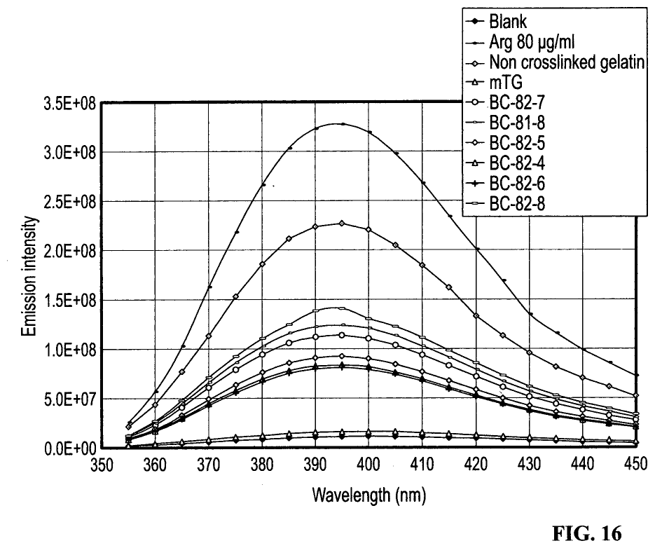

The fluorescence emissions spectra of different materials were measured and shown in FIG. 16. At a wavelength of 395 nm, the curves from top to bottom are: Arg 80 μg/ml; Non-crosslinked gelatin; BC-82-8; BC-81-8; BC-82-7; BC-82-5; BC-82-4; BC-82-6; mTG; Blank, respectively.

RGD motifs in the materials tested were quantification and calculated according to the Arginine calibration curve (FIG. 15). The free arginine used in the calibration curve had two primary amino acid groups and the RGD motif sequence had one amino groups. Calculations included the fluorescence emissions at a wavelength of 395 nm.

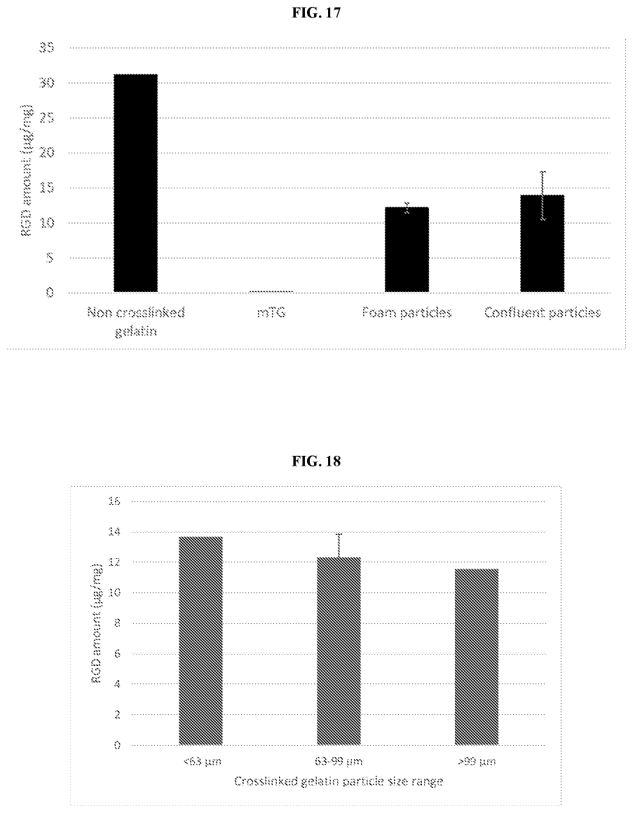

FIG. 17 shows the amount of RGD (μg/mg) in non-crosslinked gelatin, microbial transglutaminase, foam particles (FPs), and confluent particles. Non-crosslinked gelatin had over 30 μg/mg of the RGD motif, and the (FPs) and confluent particles had about 12 μg/mg and 14 μg/mg, respectively. Whereas microbial transglutaminase (mTG) essentially had zero or a barely detectable amount of RGD.

FIG. 18 shows the measured amount of RGD on cross-linked foam particles (FPs) in different size ranges.

The RGD amount (Y-axis) on FPs crosslinked with different amounts of mTG (X-axis) was also measured and shown in FIG. 19. Various batches of different gelatin to enzyme (mTG) weight ratios are illustrated on the X-axis (BC-82-7; BC-81-8; BC-82-5; BC-82-4; BC-82-6; and BC-82-8) as compared to gelatin or mTG alone.

### CONCLUSION

Increasing amounts of arginine showed a characteristic increase in emission at a wavelength of 395 nm, seen also in the gelatin raw material. mTG showed a minimal fluorescence emission spectrum at a wavelength of 395 nm. The low signal in the mTG can be due to a neglible amount of arginine by weight with respect to the total enzyme weight, indicating that quantification of RGD in the FPs was referred to as the crosslinked gelatin. This demonstrated that the RGD sequence could be quantified by using the fluorometric method.

As expected, the amount of RGD in the non-crosslinked gelatin was higher than the crosslinked gelatin microparticles, which served as a positive control in this study. The gelatin was soluble, which allowed for a large or high amount of exposed RGD sites in comparison to the insoluble crosslinked particles, in which some of the RGD sites were trapped or unexposed. Although non-crosslinked gelatin demonstrated more RGDs, use of non-crosslinked gelatin was not practical for the suggested microparticle since at 37° C., it dissolved or disintegrated very quickly with no biological effect.

A positive characteristic emission was also seen in the crosslinked gelatin microparticles, both in the confluent and in the foam particles. Though the confluent particles and the foam particles were different in their manufacturing method and in size, the RGD amount on the particle surface was similar.

FPs in different size ranges showed similar RGD amount. There was no correlation found between the amount of RGD and the amount of mTG used to crosslink the FPs. Integration of the results showed that the amount of RGD on the crosslinked gelatin particles range between 11 μg/mg-36 μg/mg.

### Example 16

### In Vitro Culture of Primary Fibroblasts on Microparticles in Suspension

Primary bovine dermal fibroblasts were incubated with microparticles of the disclosure and placed in non-tissue culture dishes. For comparison, cells were seeded in non-tissue culture dishes without microparticles, and in regular tissue culture dish. Viability was measured.

Cells: Primary bovine dermal fibroblasts (BDFs) were isolated from a 14-months old male calf, using the explant method. In the explant method, a small piece of skin, for example from the calf, was placed on a tissue culture dish until a sizable outgrowth of cells was produced. This technique was historically employed as a model of wound healing. Cells were dispersed from adherence to culture plates by washing the cell monolayer of ˜80% confluence with PBS for 5 minutes, followed by enzymatic dispersion with 0.25% trypsin for 4 minutes. For this experiment, cells at passage 5 were used.

Microparticles: Microparticles described in this disclosure, in the dry particle size range of 100 μm-700 μm were used. Microparticles were suspended for hydration in complete culture medium for 48 hours prior to incubation with cells.

Adherence: Suspended cells, ˜1×10⁵, were added to 120 mg of microparticle suspension, to a final volume of 4 ml in a 15 ml cap tube in complete culture medium. The cells and microparticles were pipetted for mixing and placed in a cell culture incubator (37° C., 5% CO₂) for two hours to allow for sufficient cell-microparticle adherence.

Seeding: The cell-microparticle suspension was gently suspended and seeded in a non-tissue culture 96-well U-shape bottom plate. BDFs in the same ratio of cells/medium volume were seeded in wells of the same plate as control. As another control, BDFs in the same ratio of cells/medium volume were seeded in wells of regular 96-well tissue culture plate.

Viability: Cell viability was measured 7 days after seeding using the Alamar Blue viability/proliferation/cytotoxicity assay (Bio-Rad). Half the medium volume in each tested well was removed and replaced with fresh medium, supplemented with 20% (v/v) of Alamar Blue reagent to a final 10% concentration. Reagent was also added to a well with medium but without cells, to serve as negative control. After 4 hours of incubation with the reagent, 60 μL medium was extracted from each test or control well and diluted 1:10 in PBS. Absorbance was measured at 570 nm and 600 nm (Shimadzu UV-1280 spectrophotometer). Viability was calculated according to the equation: ((O2×Al)−(O1×A2))/((R1×N2)−(R2×N1))*100, and expressed as percent reduction of Alamar Blue, whereas: O1 =molar extinction coefficient (E) of oxidized Alamar Blue (blue) at 570 nm, O2=E of oxidized Alamar Blue at 600 nm, R1=E of reduced Alamar Blue (red) at 570 nm, R2=E of reduced Alamar Blue at 600 nm, A1=absorbance of test wells at 570 nm, A2=absorbance of test wells at 600 nm, N1=absorbance of negative control well (media plus Alamar Blue without cells) at 570 nm, N2=absorbance of negative control well (media plus Alamar Blue without cells) at 600 nm. The culture was eventually dried fixated and analyzed by SEM for surface morphology.

Results: The viability of the cells that were adhered to the microparticles of the disclosure was comparable to that of the same number of seeded cells on a flat bottom 96-well tissue culture plate. The cells seeded in a non-tissue culture 96-well U-shape bottom plate without microparticles exhibited limited viability if any at all. SEM analysis demonstrated stretched fibroblasts with collagen fibers deposition around the cells.

### CONCLUSION

The microparticles of the disclosure provided a surface for cell adherence and support of primary bovine dermal fibroblast cells. The studies described here also demonstrated the viability of the fibroblast cells in the presence of the microparticles of the disclosure, which functioned as microcarriers in a suspension. Whereas cells incubated without the microparticles of the disclosure did not survive. See, TABLE 9.

TABLE 9
Viability of Bovine Dermal Fibroblasts (BDFs)

| | Viability (% reduction |
|---|---|
| Assay | of alamarBlue ± SD) |
| BDFs with microparticles, seeded on | 68.93 ± 12.29 |
| non-tissue culture 96-well dish | |
| BDFs without microparticles, seeded on | 22.24 ± 0.85 |
| non-tissue culture 96-well dish | |
| BDFs without microparticles, seeded on | 70.27 ± 2.86 |
| tissue culture 96-well dish | |

Primary bovine dermal fibroblast cells were isolated from a 14-months old male calf. The cell culturing occured under standard conditions (e.g., 100% relative humidity (RH), 37° C., 5% CO₂) with growth medium comprising high glucose DMEM supplemented with 10% fetal calf serum (FCS), L-glutamine, Na-pyruvate, and antibiotics and/or antimycotics. About 100,000 cells were cultured by incubating the cells with the microcarrier or plurality of microparticles of the disclosure (120 mg) in a final volume of growth medium (4 ml) for a time sufficient to allow adherence of the cells to the microcarrier, which was about two hours, resuspending the cells and microcarrier of the disclosure, and seeding the suspension in a non-tissue culture plate (e.g., 96-well U-shape bottom plate). Cells in the same ratio of cells to medium volume were seeded without microcarrier in wells of the same plate as control, and cells in the same ratio of cells to medium volume were seeded without microcarrier in wells of regular 96-well tissue culture plate wells as another control.

Viability was measured, for example, three days post-seeding, using the alamarBlue viability/proliferation/cytotoxicity assay (Bio-Rad), in accordance with the recommended manufacturer instructions. Viability (at day 3) of cells seeded on the microcarrier was similar to that of cells seeded on a tissue culture plate, while cells seeded directly on non-tissue culture plate were not viable. See, TABLE 9.

For example, 6x106 of induced pluripotent stem (iPS) cells were incubated with the FPs of the disclosure (particle size range 500 μm-2000 μm) in an incubator for an overnight on a shaker (80RPM) in an uncoated 55 mm petri dish for adhesion to the FPs. Afterward, the cell aggregates were incubated 37° C. for proliferation and differentiation for 8 days. The cell aggregate started to beat after 7 days of culture indicating that the iPS cells were differentiated successfully into cardiomyocytes and were functional.

### Example 17

### In Vitro Culturing of Induced Pluripotent Stem Cells for Cell Differentiation

iPS cells (6 million) were incubated to adhere with 100 mg of FPs (particle size range 25 μm-2000 μm) in an incubator for overnight incubation on a shaker (80 RPM) in an uncoated 55 mm petri dish. Afterward, the resulting cell aggregates were incubated at 37° C. for proliferation and differentiation for 8 days.

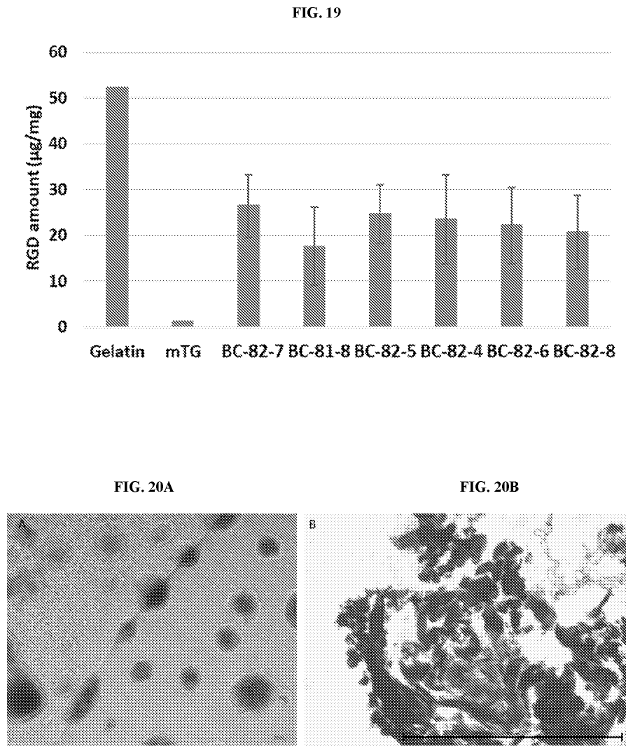

Results: The cells loaded with FPs were observed under light microscope (See, FIG. 20A; FIG. 20B). Beating of the cells loaded FPs was observed after 8 days, indicating that the iPS cells were differentiated successfully into cardiomyocytes.

### Example 18

### Foam Gelatin Particles Produced from Crosslinked Gelatin Fibers

Crosslinked gelatin was prepared from 1 g milled gelatin, with 1 g mTG. The powders were mixed and placed in a 10 ml syringe (Syringe 1). An additional syringe (Syringe 2) was filled with 8 ml saline and connected to Syringe 1. The saline of Syringe 2 was mixed with the powder of Syringe 1 through a syringe-to-syringe mixing method for 60 seconds. The produced foam was injected through various sized needles: 27G, 25G, and 21G needles into a cold (4° C.) microbial transglutaminase (mTG) solution with a concentration of 0.2% w/v placed in a petri dish and maintained at room temperature (RT) for 2 hours. Half of the petri dishes was stored for an additional 1 hour at 37° C. Finally, generated fibers were filtered from the mTG solution, dried at RT overnight, milled by mortar and pestle, and their morphology was characterized by light microscopy.

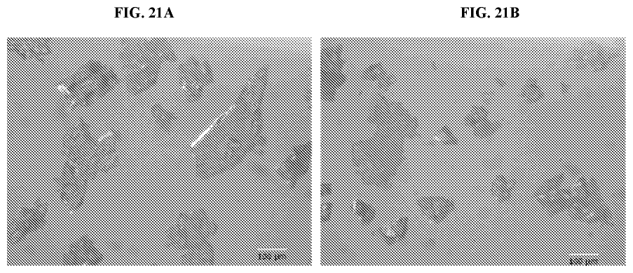

Results: light microscopy images of the particles showed that foam particles (FPs) having a particle size range of 22 μm-752 μm were successfully prepared from foam crosslinked gelatin fibers. See, FIG. 21A; FIG. 21B.

As various changes can be made in the above-described subject matter without departing from the scope and spirit of the present disclosure, it is intended that all subject matter contained in the above description, or defined in the appended claims, be interpreted as descriptive and illustrative of the present disclosure. Many modifications and variations of the present disclosure are possible in light of the above teachings. Accordingly, the present description is intended to embrace all such alternatives, modifications and variances which fall within the scope of the appended claims.

All documents cited or referenced herein and all documents cited or referenced in the herein cited documents, together with any manufacturer's instructions, descriptions, product specifications, and product sheets for any products mentioned herein or in any document incorporated by reference herein, are hereby incorporated by reference, and can be employed in the practice of the disclosure.

1-80. (canceled)

81. A plurality of microparticles, comprising:
(a) a cross-linked protein,
wherein said cross-linked protein comprises at least one RGD (Arg-Gly-Asp) motif;
wherein the plurality of microparticles is essentially cross-linker-free;
wherein the plurality of microparticles is water insoluble.

82. A composition, comprising the plurality of microparticles of claim 81.

83. The composition of claim 82, further comprising: (b) a carrier.

84. The composition of claim 82, wherein the cross-linked protein is selected from the group consisting of: gelatin, collagen, tropoelastin, elastin, casein, albumin, engineered protein thereof, any engineered polymer comprising a RGD motif, and any combinations thereof.

85. The composition of claim 82, wherein the cross-linked protein comprises the RGD motif in a range of 0.1 μg/mg-50 μg/mg.

86. The composition of claim 82, wherein the plurality of particles comprises dry foam particles or dry crosslinked gelatin-block particles.

87. The composition of claim 82, wherein the plurality of particles comprises particle sizes selected from: 0.1 μm-2000 μm.

88. The composition of claim 82, wherein the plurality of particles comprises at least two different particle sizes.

89. The composition of claim 88, wherein the at least two different particle sizes are selected from: 0.1 μm-2000 μm.

90. The composition of claim 86, wherein the particle size comprises a mean particle size of: 30 μm-500 μm.

91. The composition of claim 83, wherein the carrier is a hydrogel.

92. The composition of claim 83, wherein the carrier is selected from the group consisting of: gelatin; collagen; alginate; glycosaminoglycan (GAG); hyaluronic acid; carboxymethyl cellulose; polyethylene glycol (PEG); poly (ethylene oxide) (PEO); poly (vinyl alcohol) (PVA); poly (propylene fumarate) (PPF); polyethylene glycol (PEG); and any combinations thereof.

93. The composition of claim 83, wherein the carrier is selected from the group consisting of: uncrosslinked chondroitin sulfate polymers, uncrosslinked dermatan sulfate polymers, uncrosslinked keratan sulfate polymers, uncrosslinked heparan polymers, uncrosslinked heparan sulfate polymers, uncrosslinked hyaluronan polymers, uncrosslinked glycosaminoglycan polymers, uncrosslinked elastin and/or fibronectin, and any combinations thereof.

94. The composition of claim 83, wherein the carrier is wet or dry.

95. The composition of claim 83, wherein the composition comprises a concentration of the plurality of microparticles in the carrier of: 1 mg/ml or greater, 300 mg/ml or less, or 1 mg/ml-300 mg/ml.

96. A method of preparing the plurality of microparticles of claim 81, comprising:
(a) mixing a cross-linkable protein solution and a cross-linker solution,
wherein the cross-linkable protein solution comprises dissolving a cross-linkable protein comprising at least one RGD (Arg-Gly-Asp) motif in a liquid;
wherein the cross-linker solution comprises dissolving a cross-linker in a liquid;
(b) forming a cross-linked foam or hydrogel block comprising the mixed cross-linkable protein solution and cross-linker solution of (a);
(c) removing the cross-linker from the cross-linked foam or hydrogel block of (b) to form a cross-linker-free foam or hydrogel block; and
(d) reducing in size: the formed cross-linked foam or hydrogel block of (b), the cross-linker-free foam or hydrogel block of (c), or combinations of the formed cross-linked foam or hydrogel block of (b) and the cross-linker-free foam or hydrogel block of (c), to form a plurality of microparticles comprising size-reduced cross-linked foam of (b) and/or size-reduced cross-linker-free foam of (c).

97. The method of claim 96,
wherein the forming a cross-linked foam of (b), comprises:
(b1) whipping the cross-linkable protein solution of (a) while adding the cross-linker solution of (a) at 37° C., to form the cross-linked foam of (b) or
(b2) mixing the cross-linkable protein solution of (a) while adding the cross-linker solution of (a) at 37° C. without gas to form the cross-linked hydrogel block of (b).

98. The method of claim 96, further comprising:
(e) freezing the cross-linker-free foam or hydrogel block of (c) or plurality of particles of (d);
(f) lyophilizing the frozen cross-linker-free foam or hydrogel block of (e); and
(g) reducing in size the lyophilized cross-linker-free foam or hydrogel block of (f) to form a plurality of cross-linked foam or hydrogel particles.

99. A method of treating a subject in need of body contouring, comprising administering the composition of claim 82 at a site of the subject in need of body contouring.

100. A method of producing a cell-free protein, comprising:
growing a plurality of protein-producing cells in a cell culture comprising the plurality of microparticles of claim 81 and culture medium,
wherein growing occurs under conditions inducing protein synthesis, thereby producing a cell-free protein.
